(19) United States
(12) Patent Application Publication
(10) Pub. No.: US 2026/0193359 A1
(43) Pub. Date: Jul. 9, 2026

(54) TREATMENT OF FIBROMYALGIA

(71) Applicant: UCB BIOPHARMA SRL, Brussels (BE)

(72) Inventors: Dominique Baeten, Harmelen (NL); Andreas Goebel, Liverpool (GB); Yiannakis Christou Ioannou, Slough (GB); Marianna Lalla, London (GB); Ivan Thomas Matthews, London (GB); Paul David Meisner, Mebane, NC (US); Ruth Oliver, Slough (GB); Gustaf Erik Rydevik, Edinburgh (GB); Trevor Steven Smart, Deal (GB); Camilla Ingegerd Elisabeth Svensson, Stockholm (SE); Alexander David Vugler, Slough (GB)

(21) Appl. No.: 19/133,114

(22) PCT Filed: Nov. 27, 2023
(86) PCT No.: PCT/EP2023/083182

§ 371 Date: May 27, 2025

Related U.S. Application Data
(60) Provisional application No. 63/428,219, filed on Nov. 28, 2022.

(51) Int. Cl. C07K 16/28; A61K 31/136; A61K 31/137; A61K 31/138; A61K 31/165; A61K 31/167; A61K 31/192; A61K 31/196; A61K 31/197; A61K 31/27; A61K 31/335; A61K 31/343; A61K 31/381; A61K 31/433; A61K 31/437; A61K 31/4525; A61K 31/485; A61K 31/496; A61K 31/4985; A61K 31/519; A61K 31/55; A61K 31/5513; A61K 31/5517; A61K 31/553; A61K 31/554; A61K 39/00; A61P 29/00

(52) U.S. Cl. CPC C07K 16/283; A61K 31/136; A61K 31/137; A61K 31/138; A61K 31/165; A61K 31/167; A61K 31/192; A61K 31/196; A61K 31/197; A61K 31/27; A61K 31/335; A61K 31/343; A61K 31/381; A61K 31/433; A61K 31/437; A61K 31/4525; A61K 31/485; A61K 31/496; A61K 31/4985; A61K 31/519; A61K 31/55; A61K 31/5513; A61K 31/5517; A61K 31/553; A61K 31/554; A61P 29/00; A61K 2039/505; A61K 2039/54; A61K 2039/545; C07K 2317/24; C07K 2317/76

(57) ABSTRACT

The invention provides a method of treating or preventing fibromyalgia syndrome (FMS) in an individual, the method comprising administering to the individual a therapeutically effective amount of an FcRn antagonist, wherein the FcRn antagonist is rozanolixizumab.

### FIELD OF THE INVENTION

The present invention relates to treating fibromyalgia syndrome (FMS).

### BACKGROUND TO THE INVENTION

Fibromyalgia syndrome (FMS) is a highly prevalent (2 to 3% of adult population) and complex syndrome primarily characterized by the presence of chronic widespread pain, but which also incorporates a wide range of other symptoms adversely impacting on function and quality of life, such as severe fatigue, cognitive dysfunction, and sleep disturbances (Häuser et al., 2015). This can impair working ability, leading to reduced productivity and high healthcare costs (White et al., 2008). The direct and indirect impact of FMS is higher among patients with more severe disease (Chandran et al., 2012).

Challenges surround the diagnosis of FMS. There is no confirmatory laboratory test, and, consequently, it often takes >2 years for patients to obtain a diagnosis, often involving multiple consultations with different physicians. An absence of specific disease biomarkers also hinders patient stratification and impacts on all areas of research into FMS. Current clinical practice recommendations and guidelines for FMS include non-pharmacologic and pharmacologic strategies, with focus towards reduction of symptoms and improvement of function. Pharmacological agents include tricyclic antidepressants, anti-epileptic drugs, selective serotonin reuptake inhibitors, and norepinephrine/serotonin reuptake inhibitors. While the US Food and Drug Administration has approved pregabalin, duloxetine, and milnacipran for treatment of fibromyalgia in adults, the modest efficacy of available therapies in many patients underlines the unmet clinical need for this patient population. Patients with severe FMS are distinguished by their higher scores on pain and other scales, as well as significant increases in disability and depression (Chandran et al., 2012). Patients with severe FMS have an unmet need due to the limited treatment options available. FcRn receptor rescues IgG from intracellular lysosomal degradation by recycling it from the sorting endosome to the cell surface (Anderson et al., 2006). This is achieved by binding of IgG to the FcRn receptor. Thus, in effect FcRn salvages IgG, saving it from degradation and returning it to circulation. Albumin is similarly recycled by FcRn, though via a different binding site on the FcRn molecule. It has been shown that knockout or blockade of FcRn removes this recycling, resulting in endosomal catabolism of IgG and a marked reduction of IgG concentrations in both the vascular and extravascular (tissue) compartments. In effect, blockade of FcRn accelerates removal of endogenous IgG.

### SUMMARY OF THE INVENTION

The invention provides a method of treating or preventing fibromyalgia syndrome (FMS) in an individual, the method comprising administering to the individual a therapeutically effective amount of an FcRn antagonist, wherein the FcRn antagonist is an anti-FcRn antibody or FcRn-binding antibody fragment comprising:
(a) a heavy chain or heavy chain fragment having a variable region, wherein said variable region comprises three CDRs having the sequences given in SEQ ID NO: 4 for CDR H1, SEQ ID NO: 5 for CDR H2, and SEQ ID NO: 6 for CDR H3, and
(b) a light chain or light chain fragment having a variable region, wherein said variable region comprises three CDRs having the sequences given in SEQ ID NO: 9 for CDR L1, SEQ ID NO: 10 for CDR L2, and SEQ ID NO: 11 for CDR L3.

The FMS treated by the invention is typically severe FMS. Patients with severe FMS are distinguished by their higher scores on pain and other scales, for example wherein the individual has a pain intensity of greater than 5 and less than 10 (i.e. greater than or equal to 6 and less than 10) on the pain numeric rating scale (pain NRS) and/or a brief pain inventory-short form (BPI-SF) interference score of greater than or equal to 6. The patient may also have a Fibromyalgia Impact Questionnaire (FIQR) score of greater than or equal to 64 and/or a fatigue numeric rating scale (fatigue NRS) score of greater than or equal to 5.

The anti-FcRn antibody may be rozanolixizumab (sold under the brand name RYSTIGGO®).

The dose of anti-FcRn antibody or FcRn-binding fragment thereof is typically from 1 mg/kg to 50 mg/kg, from 4 mg/kg to 20 mg/kg, or from 7 mg/kg to 10 mg/kg. The dose may be 420 mg, 560 mg, or 840 mg. In one embodiment, the dose is 560 mg.

The anti-FcRn antibody or FcRn-binding fragment thereof may, for example, be administered once a day, three times a week, twice a week, once a week, once every two weeks, once every three weeks, once every month, once every six weeks, once every two months, once every three months, once every four months, once every five months, or once every six months.

The method may further comprise administering a therapeutically effective amount of a second agent for treating FMS, such as an analgesic, an antidepressant, a dopamine agonist, an anti-seizure drug, an antihistamine, a hypnotic, a muscle relaxant, and an antipsychotic.

### DESCRIPTION OF THE FIGURES

FIG. 1 shows that, in an open field test, administration of IgG from FMS human patients to mice resulted in a statistically significant decrease in total activity, distance moved and velocity of the mice as compared to mice administered IgG from healthy controls. Treatment of mice that had received IgG from FMS patients with an anti-mouse FcRn antibody at 30 mg/kg (UCB4470) resulted in a statistically significant increase in total activity, distance moved and velocity as compared to mice that had received the isotype control antibody (UCB101.4 30 mg/kg) and IgG from FMS patients.

FIG. 2 shows that, in a cold allodynia test, all mice showed a reduction in the latency period after treatment with antibodies (UCB101.4 or UCB4470) and administration of IgG from HC or FMS pooled samples on day 3 as compared to baseline on day 0. This reduction was similar across all groups. In mice administered IgG from FMS patients (groups FMA and FMB) that were treated with the isotype control antibody (UCB101.4) there was an overall reduction in the latency period from day 3 until day 9 compared to mice that received IgG from HC. This suggests IgG from FMS patients renders the recipient mouse hypersensitive to the cold stimulus. Treatment with UCB4470 resulted in a trend towards inhibition of this hypersensitivity when mice received IgG from the FMA pool. However, this trend was not observed with UCB4470 when mice received IgG from the FMB pool.

FIG. 3 is a schematic of a Phase 2, multi-center, randomized, double-blind, placebo-controlled, proof-of-concept study to evaluate the efficacy, safety, PK (pharmacokinetics), and PD (pharmacodynamics) of rozanolixizumab for the treatment of severe FMS. The primary endpoint is the average BPI-SF interference score after 12 weeks of double-blind treatment. EOS=end of study; QW-once weekly; sc=subcutaneous.

### DETAILED DESCRIPTION

### Introductory Comments

Current treatments for FMS, such as analgesics, focus on alleviating specific symptoms of FMS, particularly pain, without treating the underlying cause of FMS. By contrast, the present treatment is able to treat a possible cause of FMS, namely serum IgG autoantibodies. The invention inhibits binding of the IgG autoantibodies to the FcRn receptor, thereby inhibiting their salvage and accelerating their removal from the serum.

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “an antibody comprising a heavy chain” should be interpreted to mean that the antibody has a heavy chain sequence element, but the antibody may comprise further elements. In some embodiments of the invention, the word “comprising” may be replaced with the phrase “consisting of”. The term “consisting of” is intended to be limiting.

In some embodiments of the invention, the word “comprising” may be replaced with the phrase “consisting essentially of”. The term “consisting essentially of” means that specific further components can be present, namely those not materially affecting the essential characteristics of the subject matter.

For the purposes of the present invention, the term “fragment” refers to a contiguous portion of a sequence. For example, a fragment of SEQ ID NO: 1 of 50 amino acids refers to 50 contiguous amino acids of SEQ ID NO: 1.

The term “about” used in the context of describing the decrease in a subject's IgG level indicates that the decrease may be a defined percentage, plus or minus 10%, more particularly plus or minus 5%, or more particularly, plus or minus a single percentage point. For example, reference to a decrease in subject IgG levels of “about” 70% may refer to a decrease of 60-80%, more particularly a decrease of 65-75%, or more particularly a decrease of 69-71%.

The singular forms “a”, “an”, and “the” include plural referents unless the content clearly dictates otherwise. Thus, for example, reference to “an amino acid” includes two or more instances or versions of such amino acids.

All publications, patents and patent applications cited herein, whether supra or infra, are hereby incorporated by reference in their entirety.

### Fibromyalgia Syndrome (FMS)

Fibromyalgia syndrome (FMS), also called fibromyalgia, is a highly prevalent (2 to 3% of adult population) and complex syndrome primarily characterized by the presence of chronic widespread pain, but which also incorporates a wide range of other symptoms adversely impacting on function and quality of life such as severe fatigue, cognitive dysfunction, and sleep disturbances (Häuser et al., 2015). This can impair working ability, leading to reduced productivity and high healthcare costs (White et al., 2008). The direct and indirect costs related to FMS are higher among patients with more severe disease (Chandran et al., 2012).

The symptoms of FMS include pain, aching, burning sensation, stabbing pain, hyperalgesia, allodynia, stiffness, muscle spasm, fatigue, reduced sleep quality, impaired memory, attention deficit, concentration deficit, impaired speech, headache, migraine, constipation, diarrhoea, dizziness, clumsiness, anxiety, and depression.

In one embodiment, treating FMS with the methods according to the present invention comprises reducing or eliminating one or more of the symptoms of FMS.

As used herein, “treating” and “treatment” refers to any reduction in the severity of FMS and “preventing” or “prevention” refers to any reduction or delay in the onset of symptoms of FMS. One of ordinary skill in the art will appreciate that any degree of protection from, or amelioration of, FMS or symptom associated therewith is beneficial to a subject, such as a human patient. The quality of life of a patient is improved by reducing to any degree the severity of symptoms in a subject and/or delaying the appearance of symptoms.

Accordingly, the method in one aspect is performed as soon as possible after it has been determined that a subject is suffering from or at risk of suffering from FMS, in particular severe FMS.

In various aspects, the FcRn antagonist such as the FcRn antibody or FcRn-binding fragment thereof is administered via an administration regimen that achieves an improvement in BPI-SF score, FIQR score, pain NRS score, or fatigue NRS score compared to pre-treatment (baseline), wherein an improvement is a decrease in the relevant score compared to the baseline. The improvement in score may be observed at, e.g., one, two, three, four, five, six, seven, eight, nine, ten, eleven, or twelve weeks following initial administration of the antagonist. In one example the improvement in score compared to baseline is observed at 10 weeks or 12 weeks or 24 weeks after initial administration of the FcRn antagonist.

Alternatively or in addition, the FcRn antagonist, such as an FcRn antibody or FcRn-binding fragment thereof is administered via an administration regimen that achieves a reduction in IgG serum levels and/or a reduction in FMS-specific autoantibody (such as antibodies which bind to cells in the dorsal root ganglion) levels compared to pre-treatment (baseline). The reduction in levels may be observed at, e.g., one, two, three, four, five, six, seven, eight, nine, ten, eleven, twelve, or twenty-four weeks following initial administration of the antibody or antigen-binding fragment thereof. In one example the reduction in serum IgG levels is observed at 10 weeks or 12 weeks or 24 weeks after initial administration of the antibody or FcRn-binding fragment thereof. The IgG serum levels and/or FMS-specific autoantibody (such as antibodies which bind cells in the dorsal root ganglion) levels may be reduced by at least 50%, at least 55%, at least 60%, at least 65% or at least 70%, in particular compared to baseline.

### Dosage

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Generally, a therapeutically effective amount of the FcRn antagonist such as an anti-FcRn antibody of fragment thereof will be from 4 mg/kg to 50 mg/kg (e.g., 4 mg/kg to 25 mg/kg, such as about 7 mg/kg, 10 mg/kg, 15 mg/kg or 20 mg/kg). Compositions may be conveniently presented in dose forms containing a predetermined amount of an active FcRn antagonist of the disclosure per dose.

Dose ranges and regimens for any of the embodiments described herein include, but are not limited to, dosages ranging from 100 mg-4000 mg doses (such as 280 mg, 420 mg, 560 mg, 840 mg, 1120 mg or 1400 mg doses). The doses may be given every 1-10 weeks. In one embodiment, the doses are given once a week. The doses may be given by any route of administration, such as by either a subcutaneous or intravenous administration. For example, the doses may be given by subcutaneous infusion. In one embodiment, a 560 mg dose is given every 1-10 weeks.

Accordingly, the present invention provides a method of treating or preventing fibromyalgia syndrome (FMS) in a human in need thereof, the method comprising administering to the human at least three doses, preferably at least six doses of an anti-FcRn antibody or FcRn-binding fragment thereof wherein each dose is a 560 mg dose.

In one aspect of the present invention, the 560 mg doses are administered once a week, once every two weeks, once every three weeks, once every month, once every six weeks, or once every two months. Preferably, the 560 mg doses are administered once a week.

In one embodiment of the present invention, the doses are given once a week in a course of treatment lasting for one week, two week, three weeks, four weeks, five weeks, six weeks, seven weeks, eight weeks, nine weeks, ten weeks, eleven weeks, or twelve weeks. More than one course of treatment may be given. There may be a gap between the one or more courses of treatment. For example, a first weekly dose of a subsequent course of treatment may begin one month, two months, three months, four months, five months, or six months after the final dose of a previous course of weekly treatment.

In one embodiment of the present invention, the method comprises administering to a human at most 3 doses, at most 6 doses, at most 12 doses, at most 18 doses, at most 24 doses, at most 36 doses, at most 48 doses, or at most 96 doses of an anti-FcRn antagonist. In one embodiment of the present invention, the method comprises administering to a human from 3 to 48 doses, from 3 to 36 doses, from 3 to 24 doses, from 3 to 12 doses, or from 3 to 6 doses of an anti-FcRn antibody or FcRn-binding fragment thereof. In one embodiment of the present invention, the method comprises administering to a human from 6 to 48 doses, from 6 to 36 doses, from 6 to 24 doses, or from 6 to 12 doses of an anti-FcRn antibody or FcRn-binding fragment thereof.

In one embodiment of the present invention, the method comprises administering to a human one dose of an anti-FcRn antibody or antigen binding fragment thereof per week for 12 or 24 weeks, wherein each dose is a 560 mg dose. In one embodiment of the present invention, the method comprises administering to a human one dose of an anti-FcRn antibody or FcRn-binding fragment thereof per week for at least 12 weeks, wherein each dose is a 560 mg dose. The dosing may be subcutaneous.

In one embodiment, the dose is selected based on the response of the patient. For example, the dose may be selected to achieve a reduction of 30-90% (e.g. 50-80% or about 70%) in the serum IgG levels of the patient. Alternatively, the dose may be selected in order to achieve a satisfactory level of improvement in BPI-SF score, FIQR score, pain NRS score, or fatigue NRS score compared to pre-treatment (baseline). For example, where a dose does not give a satisfactory reduction in serum IgG and/or a satisfactory improvement in BPI-SF score, FIQR score, pain NRS score, or fatigue NRS score compared to pre-treatment (baseline), a higher dose may be selected. In one embodiment of the present invention, wherein a 560 mg dose does not achieve a satisfactory reduction in BPI-SF score, FIQR score, pain NRS score, or fatigue NRS score, the dose may be increased to 1.2 times, 1.5 times, 1.8 times, or 2.0 times the original dose; for example, the dose may be increased to 840 mg.

In one embodiment of the present invention, where a dose achieves a less than a 70% reduction in serum IgG levels, the dose may be increased. For example, the dose may be increased to 1.2 times, 1.5 times, 1.8 times, or 2.0 times the original dose. In one embodiment of the present invention, wherein a 560 mg dose achieves a less than 70% reduction in serum IgG levels, the dose may be increased to 840 mg.

In one embodiment of the present invention, where a dose achieves a greater than 70% reduction, the dose may be decreased. For example, the dose may be decreased to 0.75 times, 0.5 times, or 0.25 times the original dose. In one embodiment of the present invention, wherein a 560 mg dose achieves a greater than 70% reduction in IgG levels, the dose may be decreased to 420 mg.

In one example the present invention also provides a method of treating or preventing fibromyalgia syndrome (FMS) in a human in need thereof, the method comprising administering to the human at least three doses, preferably at least six doses, of an anti-FcRn antibody or antigen-binding fragment thereof wherein each dose is selected from 280 mg, 315 mg, 350 mg, 385 mg, 420 mg, 455 mg, 490 mg, 525 mg, 560 mg, 595 mg, 630 mg, 665 mg, 700 mg, 735 mg, 770 mg, 805 mg, 840 mg, 875 mg, 910 mg, 945 mg, 980 mg, 1015 mg, 1050 mg, 1085 mg and 1120 mg and wherein the anti-FcRn antibody or FcRn-binding fragment thereof optionally comprises a heavy chain comprising the sequence given in SEQ ID NO: 1 and a light chain comprising the sequence given in SEQ ID NO: 2. Again, the dose may be selected based on the weight of the patient.

In one example, doses across body weight tiers may be employed as follows (equivalent to approximately 7 mg/kg):
Body weight 40 to <49 kg: dose to be administered 280 mg
Bodyweight 49 to <69 kg: dose to be administered 420 mg
Bodyweight 69 to <89 kg: dose to be administered 560 mg
Bodyweight 89 to <109 kg; dose to be administered 700 mg
Bodyweight 109 to <129 kg; dose to be administered 840 mg
Bodyweight 129 to <149 kg; dose to be administered 980 mg
Bodyweight 149 to <169 kg; dose to be administered 1120 mg
Bodyweight ≥169 kg; dose to be administered 1260 mg

These doses may, for example, be given subcutaneously at one week intervals, for example for 12 weeks.

In another example, doses across body weight tiers may be employed as follows (equivalent to approximately 10 mg/kg):
Body weight 40 to <49 kg: dose to be administered 420 mg
Bodyweight 49 to <63 kg: dose to be administered 560 mg
Bodyweight 63 to <77 kg: dose to be administered 700 mg
Bodyweight 77 to <91 kg; dose to be administered 840 mg
Bodyweight 91 to <105 kg; dose to be administered 980 mg
Bodyweight 105 to <119 kg; dose to be administered 1120 mg
Bodyweight 119 to <133 kg; dose to be administered 1260 mg
Bodyweight 133 to <147 kg; dose to be administered 1400 mg
Bodyweight 147 to <161 kg; dose to be administered 1540 mg
Bodyweight ≥161 kg; dose to be administered 1680 mg

These doses may, for example, also be given subcutaneously at one week intervals, for example for 12 weeks.

In one example a dose equivalent to approximately 7 mg/kg is used. In one example for a body weight of between 40 and 49 kg the dose is 280 mg. In one example for a body weight of equal to or greater than 49 kg but less than 69 kg the dose is 420 mg. In one example for a body weight of equal to or greater than 69 kg but less than 89 kg the dose is 560 mg. In one example for a body weight of equal to or greater than 89 kg but less than 109 kg the dose is 700 mg. In one example for a body weight of equal to or greater than 109 kg but less than 129 kg the dose is 840 mg. In one example for a body weight of equal to or greater than 129 kg but less than 149 kg the dose is 980 mg. In one example for a body weight of equal to or greater than 149 kg but less than 169 kg the dose is 1120 mg. In one example for a body weight of equal to or greater than 169 kg the dose is 1260 mg.

Accordingly, in one example the present invention also provides a method of treating or preventing fibromyalgia syndrome (FMS) in a human in need thereof, the method comprising administering to the human at least 3 doses, preferably at least 6 doses, more preferably at least 12 doses of an anti-FcRn antibody or FcRn-binding fragment thereof wherein for a body weight of between 40 and 49 kg the dose is 280 mg, for a body weight of equal to or greater than 49 kg but less than 69 kg the dose is 420 mg, for a body weight of equal to or greater than 69 kg but less than 89 kg the dose is 560 mg, for a body weight of equal to or greater than 89 kg but less than 109 kg the dose is 700 mg, for a body weight of equal to or greater than 109 kg but less than 129 kg the dose is 840 mg, for a body weight of equal to or greater than 129 kg but less than 149 kg the dose is 980 mg, for a body weight of equal to or greater than 149 kg but less than 169 kg the dose is 1120 mg, for a body weight of equal to or greater than 169 kg the dose is 1260 mg.

Alternatively, one of the following doses across body weight tiers may be used:
Body weight ≥35 to <70 kg: dose to be administered 420 mg;
Bodyweight ≥70 kg: dose to be administered 560 mg; or
Body weight ≥35 to <70 kg: dose to be administered 420 mg
Bodyweight ≥70 to <100 kg: dose to be administered 560 mg
Bodyweight ≥100 kg: dose to be administered 840 mg; or
Body weight ≥35 to <50 kg: dose to be administered 420 mg
Bodyweight ≥50 to <100 kg: dose to be administered 560 mg
Bodyweight ≥100 kg: dose to be administered 840 mg; or
Body weight <50 kg: dose to be administered 420 mg
Bodyweight 50 kg to <100 kg: dose to be administered 560 mg
Bodyweight 100 kg: dose to be administered 840 mg; or
Body weight ≥35 to <50 kg: dose to be administered 280 mg
Bodyweight ≥50 to <70 kg: dose to be administered 420 mg
Bodyweight ≥70 to <100 kg: dose to be administered 560 mg
Bodyweight ≥100 kg: dose to be administered 840 mg
Body weight ≥35 to <50 kg: dose to be administered 420 mg
Bodyweight ≥50 to <70 kg: dose to be administered 560 mg
Bodyweight ≥70 to <100 kg: dose to be administered 840 mg
Bodyweight ≥100 kg: dose to be administered 1120 mg

In one embodiment, a patient is given a lower initial dose before subsequently being given a higher dose. For example, the following doses across body weight tiers may be used:
Body weight ≥35 to <70 kg: initial dose to be administered 280 mg, subsequent dose 420 mg;
Bodyweight ≥70 kg: initial dose to be administered 420 mg; subsequent dose 560 mg.

In one embodiment, a patient in given a higher initial dose before subsequently being given a lower dose. For example, the following doses across body weight tiers may be used:
Body weight ≥35 to <70 kg: initial dose to be administered 420 mg, subsequent dose 280 mg;
Bodyweight ≥70 kg: initial dose to be administered 560 mg; subsequent dose 420 mg.

### Severe FMS

Pain throughout the body is generally considered the most debilitating of the symptoms experienced by patients with fibromyalgia (Mease et al, 2011). The scales used to measure and monitor pain levels vary and include visual analog scales (VAS) for pain, the McGill Pain Questionnaire, the Revised Fibromyalgia Impact Questionnaire pain item (FIQR), various numerical rating scales (NRS) (often recorded in diaries), and the brief pain inventory-short form (BPI-SF).

These scales can be used to specify a patient population with severe FMS. In particular, an individual may be classed as having severe FMS if they have an average daily pain score of greater than or equal to 5 or 6 and less than 10 on the pain NRS, a BPI-SF interference score of greater than or equal to 6, an FIQR score of greater than or equal to 64, and/or a fatigue NRS score of greater than or equal to 5.

In one embodiment of the present invention, the FMS is severe FMS. In another embodiment, the individual has a pain intensity of greater than or equal to 5 or 6 and less than 10 on the pain NRS. In another embodiment, the individual has a brief pain inventory-short form (BPI-SF) interference score of greater than or equal to 6 (up to a maximum of 10). In another embodiment, the individual has a Revised Fibromyalgia Impact Questionnaire (FIQR) score of greater than or equal to 64 (up to a maximum of 100). In another embodiment, the individual has a fatigue numeric rating scale (NRS) score of greater than or equal to 5 (up to a maximum of 10). In another embodiment, the individual has a pain intensity of greater than or equal to 5 or 6 and less than 10 on the pain numeric rating scale (NRS), a brief pain inventory-short form interference score of greater than or equal to 6 (up to a maximum of 10), a Revised Fibromyalgia Impact Questionnaire (FIQR) score of greater than or equal to 64 (up to a maximum of 100), and/or a fatigue numeric rating scale (NRS) score of greater than or equal to 5 (up to a maximum of 10).

In one embodiment of the present invention, the method reduces the brief pain inventory-short form (BPI-SF) average interference score of the individual. The method may reduce the BPI-SF score of the individual by at least 1, at least 2, at least 3, at least 4, at least 5, at least 6, at least 7, at least 8, or at least 9.

In another embodiment of the present invention, the method reduces the Revised Fibromyalgia Impact Questionnaire (FIQR) score of the individual. The method may reduce the FIQR score of the individual by at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, or at least 95.

In another embodiment of the present invention, the method reduces the average daily pain score of the individual. The average daily pain score may be measured using the pain NRS. The method may reduce the average daily pain score of the individual by at least 1, at least 2, at least 3, at least 4, at least 5, at least 6, at least 7, at least 8, or at least 9.

In another embodiment of the present invention, the method reduces the fatigue score of the individual. The fatigue score may be measured using the fatigue NRS. The method may reduce the fatigue score of the individual by at least 1, at least 2, at least 3, at least 4, at least 5, at least 6, at least 7, at least 8, or at least 9.

### Mild to Moderate FMS

The FMS treated by the invention may be mild or moderate FMS. Patients with mild or moderate FMS are distinguished by their lower scores on pain and other scales, for example wherein the individual has a pain intensity of less than 5 (e.g. 2 to 4) or less than 6 (e.g. 2 to 5) on the pain numeric rating scale (pain NRS); a brief pain inventory-short form (BPI-SF) interference score of less than 6 (e.g. 2 to 5); a Fibromyalgia Impact Questionnaire (FIQR) score of less than 64 (e.g. 10 to 63, 20 to 63, 30 to 63, 40 to 63 or 50 to 63); and/or a fatigue numeric rating scale (fatigue NRS) score of less than 5 (e.g. 2 to 4).

### Brief Pain Interference-Short Form (BPI-SF)

The Brief Pain Inventory-Short Form (BPI-SF) is a 9 item self-administered questionnaire used to evaluate the severity of a patient's pain and the impact of this pain on the patient's daily functioning. The patient is asked to rate their worst, least, average, and current pain intensity, list current treatments and their perceived effectiveness, and rate the degree that pain interferes with general activity, mood, walking ability, normal work, relations with other persons, sleep, and enjoyment of life on a 10 point scale.

The BPI-SF interference score measures the impact of pain across seven domains of daily life experience and is suitable for measuring the effect of a treatment that may improve the experience of pain and the functional interference experienced by patients.

The 7 BPI-SF interference items include: general activity, mood, walking ability, normal work (including housework), relations with other people, sleep, and enjoyment of life. Each item is rated on a 0 (does not interfere) to 10 (completely interferes) scale with a recall period of 24 hours. The arithmetic mean of the 7 interference items can be used as a measure of pain interference.

### Pain NRS

The numeric rating scale is one of the most commonly used pain scales in medicine. The NRS consists of a numeric version of the visual analog scale. The most common form of the NRS is a horizontal line with an eleven point numeric range. It is labeled from zero to ten, with zero being an example of someone with no pain and ten being the worst pain possible. This type of scale can be administered verbally. It can also be administered via paper to be completed physically.

### Revised Fibromyalgia Impact Questionnaire (FIQR)

The Revised Fibromyalgia Impact Questionnaire (FIQR) is a self-reported questionnaire that measures functional capacity and disease severity in patients with fibromyalgia. The FIQR is one of the most used functional rating instruments in clinical practice and clinical trials for the evaluation of therapeutic efficacy in patients with fibromyalgia.

The FIQR consists of 21 individual questions across three linked domains: function, overall impact and symptoms. All questions pertain to experiences during the past 7 days and are scored using an 11-point numeric rating scale (0 to 10, with 10 denoting the worst possible condition). The function domain contains nine questions addressing activities of daily living and contributes to 30% of the total FIQR score weighting. The overall impact domain consists of two questions relating to the overall impact of fibromyalgia on function and overall impact of symptom severity; 20% of the FIQR total score is attributed to this domain. The symptoms domain contains 10 questions and evaluates symptoms commonly reported by fibromyalgia patients such as tenderness, hyperalgesia, environmental sensitivity, balance disturbances and memory problems; it accounts for 50% of the total FIQR score. The total FIQR score (out of 100) is the sum of the three modified domain scores, that is: the summed score for the function domain (range 0 to 90, divided by 3), the score for overall impact domain (range 0 to 20) and the score for the symptoms domain (range 0 to 100, divided by 2).

### Fatigue NRS

The fatigue NRS is a patient-administered, single-item, 11-point horizontal scale anchored at 0 and 10, with 0 representing ‘no fatigue’ and 10 representing ‘as bad as you can imagine’. Patients are asked to ‘please rate your fatigue (weariness, tiredness) by selecting the number that describes your worst level of fatigue during the past 24 hours’.

### FcRn

FcRn is a non-covalent complex of membrane protein FcRn a chain and B2 microglobulin (B2M). In adult mammals FcRn plays a key role in maintaining serum antibody levels by acting as a receptor that binds and salvages antibodies of the IgG isotype. IgG molecules are endocytosed by endothelial cells, and if they bind to FcRn, are recycled out into, for example, circulation. In contrast, IgG molecules that do not bind to FcRn enter the cells and are targeted to the lysosomal pathway where they are degraded. A variant IgG1 in which His435 is mutated to alanine results in the selective loss of FcRn-binding and a significantly reduced serum half-life (Firan et al. 2001).

### FcRn Antagonist

The FcRn antagonist may be a peptide, such as SYN1436.

The FcRn antagonist may be an Fc fragment, such as efgartigimod.

The FcRn antagonist may be an FcRn antibody or an FcRn-binding fragment thereof, such as rozanolixizumab, nipocalimab, orilanolimab, CSL730/M230, ABY-039 or RVT-1401 (HL161).

Assays suitable for assessing the ability of a test molecule such as an FcRn antagonist to block human FcRn activity and in particular the ability of human FcRn to recycle IgG are described below.

Provided herein is an assay suitable for assessing the ability of a test molecule such as an FcRn antagonist, such as an anti-FcRn antibody, to block human FcRn activity and in particular the ability of human FcRn to recycle IgG, wherein the assay comprises the steps of:
a) coating onto a surface non-human mammalian cells recombinantly expressing human FcRn alpha chain and human β2 microglobulin (2M),
b) contacting the cells under mildly acidic conditions such as about pH5.9 with a test molecule and an IgG to be recycled by the cell for a period of time sufficient to allow binding of both the test molecule and the IgG to FcRn, optionally adding the test molecule before the IgG to be recycled and incubating for a period of time sufficient to allow binding of the test molecule to FcRn.
c) washing with a slightly acidic buffer, and
d) detecting the amount of IgG internalised and/or recycled by the cells.

Provided herein is an assay suitable for assessing the ability of a test molecule such as an FcRn antagonist such as an anti-FcRn antibody, to block human FcRn activity and in particular the ability of human FcRn to recycle IgG, wherein the method comprises the steps of:
a) coating onto a surface non-human mammalian cells recombinantly expressing human FcRn alpha chain and human β2 microglobulin (2M),
b) contacting the cells under mildly acidic conditions such as about pH5.9 with a test antibody molecule and an IgG to be recycled by the cell for a period of time sufficient to allow binding of both the test antibody molecule and the IgG to FcRn, optionally adding the test antibody molecule before the IgG to be recycled and incubating for a period of time sufficient to allow binding of the test antibody molecule to FcRn.
c) washing with a slightly acidic buffer to remove unbound IgG and test antibody molecule, and
d) detecting the amount of IgG recycled by the cells.

Provided herein is an assay suitable for assessing the ability of a test molecule such as an FcRn antagonist, such as an anti-FcRn antibody, to block human FcRn activity and in particular the ability of human FcRn to recycle IgG, wherein the method comprises the steps of:
a) coating onto a surface non-human mammalian cells recombinantly expressing human FcRn alpha chain and human β2 microglobulin (β2M),
b) contacting the cells under mildly acidic conditions such as about pH5.9 with a test antibody molecule and an IgG to be recycled by the cell for a period of time sufficient to allow binding of both the test antibody molecule and IgG to FcRn, optionally adding the test antibody molecule before the IgG to be recycled and incubating for a period of time sufficient to allow binding of the test antibody molecule to FcRn.
c) washing with a slightly acidic buffer to remove unbound IgG and test antibody molecule,
d) incubating the cells in a neutral buffer such as about pH 7.2
e) detecting the amount of IgG recycled by the cells by determining the amount of IgG released into the supernatant.

Suitable cells include Madin-Darby Canine Kidney (MDCK) II cells. Transfection of MDCKII cells with human FcRn alpha chain and human β2 microglobulin (B2M) has previously been described by Claypool et al., 2002. This paper also describes recycling of IgG by these transfected cells. Media for supporting the cells during testing includes complete media comprising MEM (Gibco #21090-022), 1× non-essential amino acids (Gibco 11140-035), 1× sodium pyruvate (Gibco #11360-039), and L-glutamine (Gibco #25030-024). Acidic wash can be prepared by taking HBSS+ (PAA #H15-008) and adding 1 M MES until a pH 5.9+/−0.5 is reached. BSA about 1% may also be added (Sigma #A9647). A neutral wash can be prepared by taking HBSS+ (PAA #H15-008) and adding 10 M Hepes pH 7.2+/−0.5 is reached. BSA about 1% may also be added (Sigma #A9647). Washing the cells with acidic buffer removes the unbound test antibody and unbound IgG and allows further analysis to be performed. Acidic conditions used in step (b) encourage the binding of the IgG to FcRn and internalisation and recycling of the same.

The amount of test FcRn antagonist such as an antibody or fragment and IgG on the surface of the cells may be determined by washing the cells with neutral wash and analysing the supernatant/washings to detect the quantity of test antibody or IgG. Importantly a lysis buffer is not employed.

To determine the amount of IgG internalised by the cells the FcRn antagonist such as the antibody may first be removed from the surface of the cell with a neutral wash and the cells lysed by a lysis buffer and then the internal contents analysed. To determine the amount of IgG recycled by the cells, the cells are incubated under neutral conditions for a suitable period of time and the surrounding buffer analysed for IgG content. If the surface and internal antibody content of the cell is required then the cell can be washed with acid wash to maintain the antibody presence on the cell surface, followed by cell lysis and analysis of the combined material.

Where it is desired to measure both internalisation and recycling of the IgG samples are run in duplicate and testing for internalisation and recycling conducted separately. A suitable lysis buffer includes 150 mM NaCl, 20 mM Tris, pH 7.5, 1 mM EDTA, 1 mM EGTA, 1% Triton-X 100, for each 10 ml add protease inhibitors/phosphate inhibitors as described in manufacturer's guidelines.

Typically the IgG to be recycled is labelled. In one example a biotinylated human IgG may be used. The IgG can then be detected employing, for example a streptavidin sulfo-tag detection antibody (such as MSD #r32ad-5) 25 mL at 0.2 μg/mL of MSD blocking buffer. Blocking buffer may comprise 500 mM Tris, pH7.5. 1.5M NaCl and 0.2% Tween-20 and 1.5% BSA.

Alternatively the IgG may be pre-labelled with a fluorophore or similar label.

In one embodiment a suitable surface is a plastic plate or well such as a 96 well plate or similar, a glass slide or a membrane. In one example cells are coated onto the surface at a density that results in the formation of a monolayer.

In one embodiment the assay described herein is not a measurement of transcytosis of an antibody top to bottom across a membrane with a pH gradient there-across, for example acid conditions one side of the membrane and neutral conditions on the underside of the membrane.

In one example the test antibody or fragment and IgG may be incubated with the cells in step (b) for about 1 hour for example at ambient temperature under acidic conditions to allow binding.

In one example the test antibody or fragment may be incubated with the cells in step (b) for about 1 hour for example at ambient temperature under acidic conditions to allow binding before addition of the IgG to be recycled. Subsequently the IgG to be recycled by the cell may be incubated with the cells in step (b) for about 1 hour for example at ambient temperature under acidic conditions to allow binding.

Neutral conditions facilitate release of the IgG into the supernatant.

In an IgG recycling assay as described above, an FcRn antagonist may have an EC50 value of less than 100 nM, less than 50 nM, less than 25 nM, or less than 10 nM. For example, an FcRn antagonist may have an EC50 value of 1 to 100 nM, 1 to 50 nM, 1 to 25 nM, or 1 to 10 nM.

### Efgartigimod

Efgartigimod has been approved for generalized myasthenia gravis (MG). Efgartigimod is a monoclonal IgG1 Fc fragment that has been mutated at 5 residues to increase its affinity for FcRn at both physiologic and acidic pH. In a phase 2 randomized, placebo-controlled study in 24 patients with MG following 4 once-weekly (QW) intravenous (IV) administrations of 10 mg/kg efgartigimod, mean maximum reductions in serum IgG of 70.7% from baseline were observed 1 week after the final infusion (Peter et al., 2020).

### Nipocalimab, Orilanolimab and RVT-1401

Nipocalimab (M281), a high-affinity fully human monoclonal IgG1 anti-FcRn antibody engineered to have no Fc effector potential (no C1q binding, and no binding to activating FcgR), and orilanolimab (SYNT001), a humanized IgG4k mAb, are two additional FcRn inhibitors with published first in human (FIH) data. Nipocalimab is currently in phase 2 trials for autoimmune hemolytic anemia, hemolytic disease of the fetus and newborn, and MG.

RVT-1401 is a fully human monoclonal antibody formulated for intravenous or subcutaneous injection. A phase 1 single ascending dose/multiple ascending dose (SAD/MAD) study in healthy volunteers has been completed. The SAD portion of the study included weight-based and fixed intravenous and subcutaneous dose (fixed doses 100-765 mg), while the MAD cohorts included administration of weekly subcutaneous doses of 340 or 680 mg RVT-1401 or placebo for 4 weeks. IgG levels were reduced by 47% after single doses of 765 mg, with the nadir being reached 8-10 days after dosing. Weekly subcutaneous dosing with 680 mg reduced total IgG levels by 78%. IgG reductions ≥35% were maintained for more than 1 monthafter the last dose. Reversible dose dependent reductions in albumin were observed (31% with 680 mg subcutaneous dosing) and were asymptomatic. Single and multiple doses of RVT-1401 were well-tolerated with no subjects terminating the study early due to AEs. The most common adverse events (AEs) in the phase 1 study were injection site erythema and swelling. No subjects in the MAD cohorts developed anti-drug antibodies (Gable et al., 2020).

### FcRn Antagonist Peptides

The FcRn antagonist for use in the present invention may be a peptide. Mezo et al., 2008 describes a family of related peptides, all of which contained nine residues within a disulfide bond. The peptide may comprise a consensus sequence of Gly-His-Phe-Gly-Gly-X-Tyr, where X is preferably a hydrophobic amino acid. The consensus sequence bears no homology to the Fc domain of IgG, and the cysteine disulfide bond may be in varying positions relative to the consensus sequence.

SYN1436 is a 3.1-kDa peptide that binds to human FcRn (hFcRn) and inhibits the hFcRn-human IgG (hlgG) interaction. The core peptide sequence was discovered by using phage display peptide library screening and possesses no homology to the Fc domain of IgG. The peptide was chemically optimized to enhance its stability in vivo and binding properties for hFcRn. SYN1436 modulates IgG levels in hFcRn transgenic mice and in cynomolgus monkeys.

### IgG

In one aspect of the present invention, the administration of the FcRn antagonist results in a reduction in the individual of serum IgG of at least 10%, at least 20%, at least 30%, at least 40%, at least 50%, at least 60%, at least 70%, at least 80%, or at least 90%. The administration of the FcRn antagonist may result in a reduction in subject IgG levels of from 60% to 80%. The administration of the FcRn antagonist may result in a reduction in subject IgG levels of about 70%.

The auto-antibodies responsible for FMS may bind to satellite glial cells (SGCs) in the dorsal root ganglion (DRG). These anti-SGC IgGs may contribute to FMS (Goebel et el., 2021; Krock et al., 2022).

In one embodiment of the present invention, patients with FMS have raised serum levels of anti-SGC IgG. A patient may be selected for treatment in accordance with the invention based on a raised serum level of anti-SGC IgG. A patient with a raised serum level of anti-SGC IgG typically has severe FMS. A suitable assay for determining anti-SGC IgG levels is described in Krock et al. and below.

A human DRG can be collected. The DRG tissue may be fixed on the slides, for example with 4% PFA, blocked, for example with PBS containing 3% normal donkey serum and 0.3% Triton X-100, and may then be incubated with 100 μg/ml unconjugated anti-human IgG Fab fragments (H+L, Jackson Immunoresearch). Slides may be incubated with fibromyalgia (FM) or healthy control (HC) serum diluted 1:500 in PBS with 1% normal donkey serum and 0.1% Triton X-100. Slides may then be incubated with anti-human IgG antibodies, incubated with antibodies against glial fibrillary acidic protein (GFAP) and neurofilament 200 (NF200), then appropriate secondaries, counterstained with DAPI, and cover-slipped with Prolong Gold mounting media. Individual cells may be identified using Cellpose, a deep-learning neural network. Cellpose may be run with Python v3.7.9 and the region of interest of each cell may be imported into FIJI. Human IgG binding to SGCs and neurons may then be assessed in FIJI and the percentage of cells bound and the average integrated density of IgG binding determined. Human DRG images may be analyzed using the drgquant pipeline. Experimenters may be blinded to the serum type that cells or tissue were incubated with.

Serum from a patient with severe fibromyalgia may produce a higher percentage of IgG-bound SGCs compared to serum from a healthy control patient. For example, the serum from a patient with severe fibromyalgia may result in at least 40%, at least 50%, at least 60%, or at least 65% IgG-bound SGCs. For example, serum from a patient with severe fibromyalgia may result in 55-80% or 60-75% IgG-bound SGCs. For example, the serum from a healthy control patient may result in less than 40% IgG-bound SGCs. For example, serum from a healthy control patient may result in 20 to less than 55% IgG-bound SGCs. For example, serum from a patient with severe fibromyalgia may result in at least 1.2 times, at least 1.5 times, at least 2 times, at least 3 times or at least 4 times higher percentage IgG-bound SGCs than serum from a healthy control patient. For example, serum from a patient with severe fibromyalgia may result in a 1.2 to 5 times, 1.5 to 5 times, 2 to 5 times or 3 to 5 times higher percentage IgG-bound SGCs than serum a from healthy control patient.

Serum from a patient with severe fibromyalgia may result in an increased SGC-binding intensity compared to serum from a healthy control patient. For example, the serum from a patient with severe fibromyalgia may give a binding intensity of at least 2.5, at least 3, at least 3.5, at least 4, at least 4.5, or at least 5. For example, serum from a patient with severe fibromyalgia give a binding intensity of from 3 to 6. For example, the serum from a healthy control patient may give a binding intensity of less than 2.5, less than 2, less than 1.5 or less than 1. For example, serum from a healthy control patient may give a binding intensity of from 0 to 2.5. For example, serum from a patient with severe fibromyalgia may give a binding intensity at least 1.2 times, at least 1.5 times, at least 2 times, at least 3 times, at least 4 times or at least 5 times higher than serum from a healthy control patient. For example, serum from a patient with severe fibromyalgia may give a binding intensity 1.2 to 10 times, 1.5 to 10 times, 2 to 10 times, 3 to 10 times, 4 to 10 times or 5 to 10 times higher than serum from a healthy control patient.

In one aspect of the present invention, the administration of the FcRn antagonist results in a reduction of at least 10%, at least 20%, at least 30%, at least 40%, at least 50%, at least 60%, at least 70%, at least 80%, or at least 90 in levels of subject anti-SGC IgG. The administration of the FcRn antagonist may result in a reduction in levels of subject anti-SGC IgG of from 60% to 80%. The administration of the FcRn antagonist may result in a reduction in levels of subject ant-SGC IgG of about 70%.

### Dorsal Root Ganglion (DRG)

The 31 right and left spinal nerve pairs in humans form from afferent sensory dorsal axons (the dorsal root) and motor ventral efferent axons (the ventral root). As the dorsal root emerges from the intervertebral neural foramina, it forms the dorsal root ganglion (DRG). The DRG is a group of cell bodies responsible for the transmission of sensory messages from receptors such as thermoreceptors, nociceptors, proprioceptors, and chemoreceptors, to the CNS for a response.

The DRG contains most of the body's sensory neurons. These neurons relay sensory neural messages from the periphery to the central nervous system (brain and spinal cord). Until recently, the belief was that the DRG cell bodies merely acted as storage “helpers” in peripheral processes such as nociception. Recent studies, however, have shown that these cell bodies are not passive but rather active participants in the signalling process; they sense specific molecules and produce molecules needed to regulate the process.

The DRG has a significant clinical application, particularly in its association with neuropathic pain. DRG neurons emerge from the dorsal root of the spinal nerves, carrying sensory messages from various receptors, including those for pain and temperature towards the central nervous system for a response.

In addition to neurons, the DRG also contains SGCs which form thin cellular sheaths covering the neurons. SGCs perform a number of roles linked to their close interactions with neurons, including regulating the microenvironment surrounding neurons. SGCs express a variety of receptors which regulate the levels of neurotransmitters in the neuron microenvironment, and thereby influence excitation of the neuron. IgG binding to SGCs may therefore influence nociception in the DRG.

Until recently, the dorsal root ganglion has been considered a passive organ that metabolically assists functions and pathways between the peripheral nervous system (PNS) and central nervous system (CNS). New studies suggest, however, that the DRG is an active participant in peripheral processes, including Paroxysmal Atrial Fibrillation (PAF) injury, inflammation, and neuropathic pain development. A proper understanding of the significance and functioning of the DRG can help improve the diagnosis and treatment of neuropathic pain syndromes, such as fibromyalgia syndrome (FMS). (Ahimsadasan et al., Neuroanatomy, Dorsal Root Ganglion)

### Antibody

The term ‘antibody’ as used herein generally relates to intact (whole) antibodies i.e. comprising the elements of two heavy chains and two light chains. The antibody may comprise further additional binding domains for example as per the molecule DVD-Ig as disclosed in WO 2007/024715, or the so-called (FabFv) 2Fc described in WO2011/030107. Thus antibody as employed herein includes bi, tri or tetra-valent full length antibodies.

Binding fragments of antibodies include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, tribodies, triabodies, tetrabodies and epitope-binding fragments of any of the above (see for example Holliger and Hudson, 2005; Adair and Lawson, 2005). The methods for creating and manufacturing these antibody fragments are well known in the art (see for example Verma et al., 1998). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170 and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583.

A typical Fab′ molecule comprises a heavy and a light chain pair in which the heavy chain comprises a variable region VH, a constant domain CH1 and a natural or modified hinge region and the light chain comprises a variable region VL and a constant domain CL.

In one embodiment there is provided a dimer of a Fab′ according to the present disclosure to create a F(ab′)2 for example dimerisation may be through the hinge.

In one embodiment the antibody or binding fragment thereof comprises a binding domain. A binding domain will generally comprises 6 CDRs, three from a heavy chain and three from a light chain. In one embodiment the CDRs are in a framework and together form a variable region. Thus in one embodiment an antibody or binding fragment comprises a binding domain specific for antigen comprising a light chain variable region and a heavy chain variable region.

It will be appreciated that one or more (for example 1, 2, 3 or 4) amino acid substitutions, additions and/or deletions may be made to the CDRs or other sequences (e.g variable domains) provided by the present disclosure without significantly altering the ability of the antibody to bind to FcRn. The effect of any amino acid substitutions, additions and/or deletions can be readily tested by one skilled in the art, for example by using the methods described herein, in particular in the Examples, to determine FcRn.

In one or more (for example 1, 2, 3 or 4) amino acid substitutions, additions and/or deletions may be made to the framework region employed in the antibody or fragment provided by the present disclosure and wherein binding affinity to FcRn is retained or increased.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia, 1987, the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

Antibodies and fragments of the present disclosure block FcRn and may thereby prevent it functioning in the recycling of IgG. Blocking as employed herein refers to physically blocking such as occluding the receptor but will also include where the antibody or fragments binds an epitope that causes, for example a conformational change which means that the natural ligand to the receptor no longer binds. Antibody molecules of the present disclosure bind to FcRn and thereby decrease or prevent (e.g. inhibit) FcRn binding to an IgG constant region.

In one embodiment the antibody or fragment thereof binds FcRn competitively with respect to IgG. In one example the antibody or binding fragment thereof functions as a competitive inhibitor of human FcRn binding to human IgG. In one example the antibody or binding fragment thereof binds to the IgG binding site on FcRn. In one example the antibody or binding fragment thereof does not bind β2M.

Antibodies for use in the present disclosure may be obtained using any suitable method known in the art. The FcRn polypeptide/protein including fusion proteins, cells (recombinantly or naturally) expressing the polypeptide (such as activated T cells) can be used to produce antibodies which specifically recognise FcRn. The polypeptide may be the ‘mature’ polypeptide or a biologically active fragment or derivative thereof. The human protein is registered in Swiss-Prot under the number P55899. The extracellular domain of human FcRn alpha chain is provided in SEQ ID NO:21. The sequence of β2M is provided in SEQ ID NO:22.

In one embodiment the antigen is a mutant form of FcRn which is engineered to present FcRn on the surface of a cell, such that there is little or no dynamic processing where the FcRn is internalised in the cell, for example this can be achieved by making a mutation in the cytoplasmic tail of the FcRn alpha chain, wherein di-leucine is mutated to di-alanine as described in Ober et al 2001.

Polypeptides, for use to immunize a host, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The FcRn polypeptide may in some instances be part of a larger protein such as a fusion protein for example fused to an affinity tag or similar.

Antibodies generated against the FcRn polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975), the trioma technique, the human B-cell hybridoma technique (Kozbor et al., 1983) and the EBV-hybridoma technique (Cole et al., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996; WO92/02551; WO2004/051268 and International Patent Application number WO2004/106377.

Screening for antibodies can be performed using assays to measure binding to human FcRn and/or assays to measure the ability to block IgG binding to the receptor. An example of a binding assay is an ELISA, in particular, using a fusion protein of human FcRn and human Fc, which is immobilized on plates, and employing a secondary antibody to detect anti-FcRn antibody bound to the fusion protein. Examples of suitable antagonistic and blocking assays are described in the Examples herein.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005). Humanised antibodies may optionally further comprise one or more framework residues derived from the non-human species from which the CDRs were derived. The latter are often referred to as donor residues.

Specific as employed herein is intended to refer to an antibody that only recognises the antigen to which it is specific or an antibody that has significantly higher binding affinity to the antigen to which it is specific compared to binding to antigens to which it is non-specific, for example at least 5, 6, 7, 8, 9, 10 times higher binding affinity. Binding affinity may be measured by techniques such as BIAcore as described herein below. In one example the antibody of the present disclosure does not bind β2 microglobulin (β2M). In one example the antibody of the present disclosure binds cynomolgus FcRn. In one example the antibody of the present disclosure does not bind rat or mouse FcRn.

In one embodiment the antibody or fragments according to the disclosure are humanised. As used herein, the term ‘humanised antibody molecule’ refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody). For a review, see Vaughan et al., 1998. In one embodiment rather than the entire CDR being transferred, only one or more of the specificity determining residues from any one of the CDRs described herein above are transferred to the human antibody framework (see for example, Kashmiri et al., 2005). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions.

Suitably, the humanised antibody according to the present disclosure has a variable domain comprising human acceptor framework regions as well as one or more of the CDRs provided specifically herein. Thus, provided in one embodiment is blocking humanised antibody which binds human FcRn wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody of the present disclosure, the acceptor heavy and light chains do not necessarily need to be derived from the same antibody and may, if desired, comprise composite chains having framework regions derived from different chains.

One such suitable framework region for the heavy chain of the humanised antibody of the present disclosure is derived from the human sub-group VH3 sequence 1-3 3-07 together with JH4 (SEQ ID NO: 3).

Accordingly, in one example there is provided a humanised antibody comprising the sequence given in SEQ ID NO: 4 for CDR-H1, the sequence given in SEQ ID NO: 5 for CDR-H2 and the sequence given in SEQ ID NO: 6 for CDRH3, wherein the heavy chain framework region is derived from the human subgroup VH3 sequence 1-3 3-07 together with JH4.

The sequence of human JH4 is as follows: (YFDY) WGQGTLVTVS (Seq ID No: 7). The YFDY motif is part of CDR-H3 and is not part of framework 4 (Ravetch, J V. et al., 1981, Cell, 27, 583-591).

In one example the heavy chain variable domain of the antibody comprises the sequence given in SEQ ID NO: 1.

A suitable framework region for the light chain of the humanised antibody of the present disclosure is derived from the human germline sub-group VK1 sequence 2-1-(1) A30 together with JK2 (SEQ ID NO: 8).

Accordingly, in one example there is provided a humanised antibody comprising the sequence given in SEQ ID NO: 9 for CDR-L1, the sequence given in SEQ ID NO: 10 for CDR-L2 and the sequence given in SEQ ID NO: 11 for CDRL3, wherein the light chain framework region is derived from the human subgroup VK1 sequence 2-1-(1) A30 together with JK2.

The JK2 sequence is as follows: (YT) FGQGTKLEIK (Seq ID No: 12). The YT motif is part of CDR-L3 and is not part of framework 4 (Hieter, PA., et al., 1982).

In one example the light chain variable domain of the antibody comprises the sequence given in SEQ ID NO: 2.

In a humanised antibody of the present disclosure, the framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998). Such changes should be kept to the minimum necessary to recover the affinity of the donor antibody. A protocol for selecting residues in the acceptor framework regions which may need to be changed is set forth in WO91/09967.

Thus in one embodiment 1, 2, 3, 4, or 5 residues in the framework are replaced with an alternative amino acid residue. Accordingly, in one example there is provided a humanised antibody, wherein at least the residues at each of positions 3, 24, 76, 93 and 94 of the variable domain of the heavy chain (Kabat numbering) are donor residues, see for example the sequence given in SEQ ID NO: 1.

In one embodiment residue 3 of the heavy chain variable domain is replaced with an alternative amino acid, for example glutamine.

In one embodiment residue 24 of the heavy chain variable domain is replaced with an alternative amino acid, for example alanine.

In one embodiment residue 76 of the heavy chain variable domain is replaced with an alternative amino acid, for example asparagine.

In one embodiment residue 93 of the heavy chain is replaced with an alternative amino acid, for example alanine.

In one embodiment residue 94 of the heavy chain is replaced with an alternative amino acid, for example arginine.

In one embodiment residue 3 is glutamine, residue 24 is alanine, residue 76 is aspargine, residue 93 is alanine and residue 94 is arginine in the humanised heavy chain variable region according to the present disclosure.

Accordingly, in one example there is provided a humanised antibody, wherein at least the residues at each of positions 36, 37 and 58 of the variable domain of the light chain (Kabat numbering) are donor residues, see for example the sequence given in SEQ ID NO: 2.

In one embodiment residue 36 of the light chain variable domain is replaced with an alternative amino acid, for example tyrosine.

In one embodiment residue 37 of the light chain variable domain is replaced with an alternative amino acid, for example glutamine.

In one embodiment residue 58 of the light chain variable domain is replaced with an alternative amino acid, for example valine.

In one embodiment residue 36 is tyrosine, residue 37 is glutamine and residue 58 is valine, in the humanised heavy chain variable region according to the present disclosure.

In one embodiment the disclosure provides an antibody sequence which is 80% similar or identical to a sequence disclosed herein, for example 85%, 90%, 91%, 92%, 93%, 94%, 95% 96%, 97%, 98% or 99% over part or whole of the relevant sequence, for example a variable domain sequence, a CDR sequence or a variable domain sequence, excluding the CDRs. In one embodiment the relevant sequence is SEQ ID NO: 2. In one embodiment the relevant sequence is SEQ ID NO: 1.

In one embodiment, the present disclosure provides an antibody molecule which binds human FcRn comprising a heavy chain, wherein the variable domain of the heavy chain comprises a sequence having at least 80%, 85%, 90%, 91%, 92%, 93%, 94%, 95% 96%, 97%, 98% or 99% identity or similarity to the sequence given in SEQ ID NO:1.

In one embodiment, the present disclosure provides an antibody molecule which binds human FcRn comprising a light chain, wherein the variable domain of the light chain comprises a sequence having at least 80%, 85%, 90%, 91%, 92%, 93%, 94%, 95% 96%, 97%, 98% or 99% identity or similarity to the sequence given in SEQ ID NO:2.

In one embodiment the present disclosure provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO: 1 but wherein the antibody molecule has the sequence given in SEQ ID NO: 4 for CDR-H1, the sequence given in SEQ ID NO: 5 for CDR-H2 and the sequence given in SEQ ID NO: 6 for CDR-H3.

In one embodiment the present disclosure provides an antibody molecule which binds human FcRn wherein the antibody has a light chain variable domain which is at least 90%, 91%, 92%, 93%, 94%, 95% 96%, 97%, 98% or 99% similar or identical to the sequence given in SEQ ID NO: 2 but wherein the antibody molecule has the sequence given in SEQ ID NO: 9 for CDR-L1, the sequence given in SEQ ID NO: 10 for CDR-L2 and the sequence given in SEQ ID NO: 11 for CDR-L3.

In one embodiment the present disclosure provides an antibody molecule which binds human FcRn wherein the antibody has a heavy chain variable domain which is at least 90%, 91%, 92%, 93%, 94%, 95% 96%, 97%, 98% or 99% similar or identical to the sequence given in SEQ ID NO:1 and a light chain variable domain which is at least 90%, 91%, 92%, 93%, 94%, 95% 96%, 97%, 98% or 99% similar or identical to the sequence given in SEQ ID NO:2 but wherein the antibody molecule has the sequence given in SEQ ID NO: 4 for CDR-H1, the sequence given in SEQ ID NO: 5 for CDR-H2, the sequence given in SEQ ID NO: 6 for CDR-H3, the sequence given in SEQ ID NO: 9 for CDR-L1, the sequence given in SEQ ID NO: 10 for CDR-L2 and the sequence given in SEQ ID NO: 11 for CDR-L3.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656,).

The antibody molecules of the present disclosure may comprise a complete antibody molecule having full length heavy and light chains or a fragment thereof and may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Adair and Lawson, 2005). The methods for creating and manufacturing these antibody fragments are well known in the art (see for example Verma et al., 1998). Other antibody fragments for use in the present disclosure include the Fab and Fab′ fragments described in International patent applications WO2005/003169, WO2005/003170 and WO2005/003171. Multi-valent antibodies may comprise multiple specificities e.g bispecific or may be monospecific (see for example WO 92/22853, WO05/113605, WO2009/040562 and WO2010/035012).

In one embodiment the antibody molecule of the present disclosure is an antibody Fab fragment comprising the variable regions shown in SEQ ID NOs: 2 and 1 for example for the light and heavy chain respectively. In one embodiment the antibody molecule has a light chain comprising the sequence given in SEQ ID NO: 13 and a heavy chain comprising the sequence given in SEQ ID NO: 14.

In one embodiment the antibody molecule of the present disclosure is a full length IgG1 antibody comprising the variable regions shown in SEQ ID NOs: 2 and 1 for example for the light and heavy chain respectively. In one embodiment the antibody molecule has a light chain comprising the sequence given in SEQ ID NO: 13 and a heavy chain comprising the sequence given in SEQ ID NO: 15.

In one embodiment the antibody molecule of the present disclosure is a full length IgG4 format comprising the variable regions shown in SEQ ID NOs: 2 and 1 for example for the light and heavy chain respectively. In one embodiment the antibody molecule has a light chain comprising the sequence given in SEQ ID NO: 13 and a heavy chain comprising the sequence given in SEQ ID NO: 16.

In one embodiment the antibody molecule of the present disclosure is a full length IgG4P format comprising the variable regions shown in SEQ ID NOs: 2 and 1 for example for the light and heavy chain respectively. In one embodiment the antibody molecule has a light chain comprising the sequence given in SEQ ID NO: 13 and a heavy chain comprising the sequence given in SEQ ID NO: 17.

IgG4P as employed herein is a mutation of the wild-type IgG4 isotype where amino acid 241 is replaced by proline see for example where serine at position 241 has been changed to proline as described in Angal et al., 1993.

In one embodiment the antibody according to the present disclosure is provided as FcRn-binding antibody fusion protein which comprises an immunoglobulin moiety, for example a Fab or Fab′ fragment, and one or two single domain antibodies (dAb) linked directly or indirectly thereto, for example as described in WO2009/040562, WO2010035012, WO2011/030107, WO2011/061492 and WO2011/086091 all incorporated herein by reference.

In one embodiment the fusion protein comprises two domain antibodies, for example as a variable heavy (VH) and variable light (VL) pairing, optionally linked by a disulphide bond.

In one embodiment the Fab or Fab′ element of the fusion protein has the same or similar specificity to the single domain antibody or antibodies. In one embodiment the Fab or Fab′ has a different specificity to the single domain antibody or antibodies, that is to say the fusion protein is multivalent. In one embodiment a multivalent fusion protein according to the present disclosure has an albumin binding site, for example a VH/VL pair therein provides an albumin binding site. In one such embodiment the heavy chain comprises the sequence given in SEQ ID NO: 18 and the light chain comprises the sequence given in SEQ ID NO: 19 or SEQ ID NO: 20.

In one embodiment the Fab or Fab′ according to the present disclosure is conjugated to a PEG molecule or human serum albumin.

CA170_01519g57 and 1519 and 1519.g57 are employed interchangeably herein and are used to refer to a specific pair of antibody variable regions which may be used in a number of different formats. These variable regions are the heavy chain sequence given in SEQ ID NO: 1 and the light chain sequence given in SEQ ID NO: 2.

The constant region domains of the antibody molecule of the present disclosure, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example IgG4 molecules in which the serine at position 241 has been changed to proline as described in Angal et al., 1993 may be used. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, 1995). Accordingly, the C-terminal lysine of the antibody heavy chain may be absent.

In one embodiment the antibody heavy chain comprises a CH1 domain and the antibody light chain comprises a CL domain, either kappa or lambda.

In one embodiment the light chain has the sequence given in SEQ ID NO: 13 and the heavy chain has the sequence given in SEQ ID NO: 17.

In one embodiment the light chain has the sequence given in SEQ ID NO: 13 and the heavy chain has the sequence given in SEQ ID NO: 15.

In one embodiment a C-terminal amino acid from the antibody molecule is cleaved during post-translation modifications.

In one embodiment an N-terminal amino acid from the antibody molecule is cleaved during post-translation modifications.

Also provided by the present disclosure is a specific region or epitope of human FcRn which is bound by an antibody provided by the present disclosure, in particular an antibody comprising the heavy chain sequence gH20 (SEQ ID NO: 1) and/or the light chain sequence gL20 (SEQ ID NO: 2).

This specific region or epitope of the human FcRn polypeptide can be identified by any suitable epitope mapping method known in the art in combination with any one of the antibodies provided by the present disclosure. Examples of such methods include screening peptides of varying lengths derived from FcRn for binding to the antibody of the present disclosure with the smallest fragment that can specifically bind to the antibody containing the sequence of the epitope recognised by the antibody. The FcRn peptides may be produced synthetically or by proteolytic digestion of the FcRn polypeptide. Peptides that bind the antibody can be identified by, for example, mass spectrometric analysis. In another example, NMR spectroscopy or X-ray crystallography can be used to identify the epitope bound by an antibody of the present disclosure. Once identified, the epitopic fragment which binds an antibody of the present disclosure can be used, if required, as an immunogen to obtain additional antibodies which bind the same epitope.

In one embodiment the antibody of the present disclosure binds the human FcRn alpha chain extracellular sequence as shown below:

(SEQ ID NO: 21)
AESHLSLLYH LTAVSSPAPG TPAFWVSGWL GPQQYLSYNS
LRGEAEPCGA WVWENQVSWY WEKETTDLRI KEKLFLEAFK
ALGGKGPYTL QGLLGCELGP DNTSVPTAKF ALNGEEFMNF
DLKQGTWGGD WPEALAISQR WQQQDKAANK ELTFLLFSCP
HRLREHLERG RGNLEWKEPP SMRLKARPSS PGFSVLTCSA
FSFYPPELQL RFLRNGLAAG TGQGDEGPNS DGSFHASSSL
TVKSGDEHHY CCIVQHAGLA QPLRVELESP AKSS.

The residues underlined are those known to be critical for the interaction of human FcRn with the Fc region of human IgG and those residues highlighted in bold and italics are those involved in the interaction of FcRn with the 1519 antibody of the present disclosure comprising the heavy chain sequence gH20 (SEQ ID NO: 1) and the light chain sequence gL20 (SEQ ID NO: 2).

In one example, the present disclosure provides an anti-FcRn antibody molecule which binds an epitope of human FcRn which comprises at least one amino acid selected from the group consisting of residues V105, P106, T107, A108 and K109 of SEQ ID NO: 21 and at least one residue, for example at least 2, 3, 4, 5, 6, 7, 8, 9 or 10 residues selected from the group consisting of P100, E115, E116, F117, M118, N119, F120, D121, L122, K123, Q124, G128, G129, D130, W131, P132 and E133 of SEQ ID NO:94.

In one example the epitope of the antibody molecule is determined by X-ray crystallography using the FcRn alpha chain extracellular sequence (SEQ ID NO: 21) in complex with β2M.

In one example, the present disclosure provides an anti-FcRn antibody molecule which binds an epitope of human FcRn which comprises at least one amino acid selected from the group consisting of residues V105, P106, T107, A108 and K109 of SEQ ID NO: 21 and at least one residue, for example at least 2, 3, 4, 5, 6, 7, 8, 9 or 10 residues, selected from the group consisting of E115, E116, F117, M118, N119, F120, D121, L122, K123 and Q124 of SEQ ID NO: 21.

In one example, the present disclosure provides an anti-FcRn antibody molecule which binds an epitope of human FcRn which comprises at least two, three, four or five amino acids selected from the group consisting of residues V105, P106, T107, A108 and K109 of SEQ ID NO: 21 and at least one residue selected from the group consisting of E115, E116, F117, M118, N119, F120, D121, L122, K123 and Q124 of SEQ ID NO: 21.

In one example, the present disclosure provides an anti-FcRn antibody molecule which binds an epitope of human FcRn which comprises at least one amino acid selected from the group consisting of residues V105, P106, T107, A108 and K109 of SEQ ID NO: 21 and at least one residue selected from the group consisting of P100, E115, E116, F117, M118, N119, F120, D121, L122, K123, Q124, G128, G129, D130, W131, P132 and E133 of SEQ ID NO: 21.

In one example, the present disclosure provides an anti-FcRn antibody molecule which binds an epitope of human FcRn which comprises at least one amino acid selected from the group consisting of residues V105, P106, T107, A108 and K109 of SEQ ID NO: 21 and at least one residue selected from the group consisting of P100, M118, N119, F120, D121, L122, K123, Q124 and G128 of SEQ ID NO: 21.

In one example, the present disclosure provides an anti-FcRn antibody molecule which binds an epitope of human FcRn which comprises residues V105, P106, T107, A108 and K109 of SEQ ID NO: 21 and at least one residue selected from the group consisting of P100, M118, N119, F120, D121, L122, K123, Q124 and G128 of SEQ ID NO: 21.

In one example, the present disclosure provides an anti-FcRn antibody molecule which binds an epitope of human FcRn which comprises residues V105, P106, T107, A108 and K109 of SEQ ID NO: 21 and at least one residue selected from the group consisting of P100, E115, E116, F117, M118, N119, F120, D121, L122, K123, Q124, G128, G129, D130, W131, P132 and E133 of SEQ ID NO: 21.

In one example, the present disclosure provides an anti-FcRn antibody molecule which binds an epitope of human FcRn which comprises residues P100, V105, P106, T107, A108 and K109 of SEQ ID NO: 21 and at least one residue selected from the group consisting of E115, E116, F117, M118, N119, F120, D121, L122, K123, Q124, G128, G129, D130, W131, P132 and E133 of SEQ ID NO: 21.

In one example ‘at least one residue’ may be 1, 2, 3, 4, 5, 6, 7, 8, 9, 10, 11, 12, 13, 14, 15 or 16 residues.

In one example the present disclosure provides an anti-FcRn antibody molecule which binds an epitope of human FcRn which comprises or consists of residues 100, 105 to 109, 115 to 124 and 129 to 133 of SEQ ID NO: 21.

Antibodies which cross-block the binding of an antibody molecule according to the present disclosure in particular, an antibody molecule comprising the heavy chain sequence given in SEQ ID NO: 1 and the light chain sequence given in SEQ ID NO: 2 may be similarly useful in blocking FcRn activity. Accordingly, the present disclosure also provides an anti-FcRn antibody molecule, which cross-blocks the binding of any one of the antibody molecules described herein above to human FcRn and/or is cross-blocked from binding human FcRn by any one of those antibodies. In one embodiment, such an antibody binds to the same epitope as an antibody described herein above. In another embodiment the cross-blocking neutralising antibody binds to an epitope which borders and/or overlaps with the epitope bound by an antibody described herein above.

Cross-blocking antibodies can be identified using any suitable method in the art, for example by using competition ELISA or BIAcore assays where binding of the cross blocking antibody to human FcRn prevents the binding of an antibody of the present disclosure or vice versa. Such cross blocking assays may use isolated natural or recombinant FcRn or a suitable fusion protein/polypeptide. In one example binding and cross-blocking is measured using recombinant human FcRn extracellular domain (SEQ ID NO: 21). In one example the recombinant human FcRn alpha chain extracellular domain is used in a complex with β2 microglobulin (β2M) (SEQ ID NO: 22).

In one embodiment there is provided an anti-FcRn antibody molecule which blocks FcRn binding to IgG and which cross-blocks the binding of an antibody whose heavy chain comprises the sequence given in SEQ ID NO: 1 and whose light chain comprises the sequence given in SEQ ID NO: 2 to human FcRn. In one embodiment the cross-blocking antibodies provided by the present disclosure inhibit the binding of an antibody comprising the heavy chain sequence given in SEQ ID NO: 1 and the light chain sequence given in SEQ ID NO: 2 by greater than 80%, for example by greater than 85%, such as by greater than 90%, in particular by greater than 95%.

Alternatively or in addition, anti-FcRn antibodies according to this aspect of the disclosure may be cross-blocked from binding to human FcRn by an antibody comprising the heavy chain sequence given in SEQ ID NO: 1 and the light chain sequence given in SEQ ID NO: 2. Also provided therefore is an anti-FcRn antibody molecule which blocks FcRn binding to IgG and which is cross-blocked from binding human FcRn by an antibody comprising the heavy chain sequence given in SEQ ID NO: 1 and the light chain sequence given in SEQ ID NO: 2. In one embodiment the anti-FcRn antibodies provided by this aspect of the disclosure are inhibited from binding human FcRn by an antibody comprising the heavy chain sequence given in SEQ ID NO: 1 and the light chain sequence given in SEQ ID NO: 2 by greater than 80%, for example by greater than 85%, such as by greater than 90%, in particular by greater than 95%.

In one embodiment the cross-blocking antibodies provided by the present disclosure are fully human. In one embodiment the cross-blocking antibodies provided by the present disclosure are humanised. In one embodiment the cross-blocking antibodies provided by the present disclosure have an affinity for human FcRn of 100 μM or less. In one embodiment the cross-blocking antibodies provided by the present disclosure have an affinity for human FcRn of 50 μM or less. Affinity can be measured using the methods described herein below.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or solvent accessible surface thereof carries no net electrical charge. In one example, the FcRn antibody and fragments of the disclosure may be engineered to have an appropriate isoelectric point. This may lead to antibodies and/or fragments with more robust properties, in particular suitable solubility and/or stability profiles and/or improved purification characteristics.

Thus in one aspect the disclosure provides a humanised FcRn antibody engineered to have an isoelectric point different to that of the originally identified antibody. The antibody may, for example be engineered by replacing an amino acid residue such as replacing an acidic amino acid residue with one or more basic amino acid residues. Alternatively, basic amino acid residues may be introduced or acidic amino acid residues can be removed. Alternatively, if the molecule has an unacceptably high pI value acidic residues may be introduced to lower the pI, as required. It is important that when manipulating the pI care must be taken to retain the desirable activity of the antibody or fragment. Thus in one embodiment the engineered antibody or fragment has the same or substantially the same activity as the “unmodified” antibody or fragment.

Programs such as ExPASY http://www.expasy.ch/tools/pi_tool.html, and http://www.iut-arles.up.univ-mrs.fr/w3bb/d_abim/compo-p.html, may be used to predict the isoelectric point of the antibody or fragment.

The antibody molecules of the present disclosure suitably have a high binding affinity, in particular in the nanomolar range. Affinity may be measured using any suitable method known in the art, including BIAcore, as described in the Examples herein, using isolated natural or recombinant FcRn or a suitable fusion protein/polypeptide. In one example affinity is measured using the recombinant human FcRn alpha chain extracellular domain (SEQ ID NO: 21) in association with β2 microglobulin (β2M) (SEQ ID NO: 22). Suitably the antibody molecules of the present disclosure have a K_{D} for isolated human FcRn of about 1 nM or lower. In one embodiment the antibody molecule of the present disclosure has a K_{D} of about 500 pM or lower (i.e. higher affinity). In one embodiment the antibody molecule of the present disclosure has a K_{D} of about 250 pM or lower. In one embodiment the antibody molecule of the present disclosure has a K_{D} of about 200 pM or lower. In one embodiment the present disclosure provides an anti-FcRn antibody with a K_{D} of about 100 pM or lower. In one embodiment the present disclosure provides a humanised anti-FcRn antibody with a K_{D} of about 100 pM or lower. In one embodiment the present disclosure provides an anti-FcRn antibody with a K_{D} of 50 pM or lower.

Importantly the antibodies of the present disclosure are able to bind human FcRn at both pH6 and pH 7.4 with comparable binding affinity. Advantageously therefore the antibodies are able to continue to bind FcRn even within the endosome, thereby maximising the blocking of FcRn binding to IgG.

In one embodiment the present disclosure provides an anti-FcRn antibody with a K_{D} of 100 pM or lower when measured at pH 6 and pH 7.4.

The affinity of an antibody or binding fragment of the present disclosure, as well as the extent to which a binding FcRn antagonist (such as an antibody) inhibits binding, can be determined by one of ordinary skill in the art using conventional techniques, for example those described by Scatchard et al., 1949 or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, 1993).

In the present disclosure affinity of the test antibody molecule is typically determined using SPR as follows. The test antibody molecule is captured on the solid phase and human FcRn alpha chain extracellular domain in non-covalent complex with β2M is run over the captured antibody in the mobile phase and affinity of the test antibody molecule for human FcRn determined. The test antibody molecule may be captured on the solid phase chip surface using any appropriate method, for example using an anti-Fc or anti Fab′ specific capture FcRn antagonist. In one example the affinity is determined at pH 6. In one example the affinity is determined at pH 7.4.

It will be appreciated that the affinity of antibodies provided by the present disclosure may be altered using any suitable method known in the art. The present disclosure therefore also relates to variants of the antibody molecules of the present disclosure, which have an improved affinity for FcRn. Such variants can be obtained by a number of affinity maturation protocols including mutating the CDRs (Yang et al., 1995), chain shuffling (Marks et al., 1992), use of mutator strains of E. coli (Low et al., 1996), DNA shuffling (Patten et al., 1997), phage display (Thompson et al., 1996) and sexual PCR (Crameri et al., Nature, 1998). Vaughan et al. (supra) discusses these methods of affinity maturation.

In one embodiment the antibody molecules of the present disclosure block human FcRn activity. Assays suitable for determining the ability of an antibody to block FcRn are described in the Examples herein. Suitable assays for determining whether antibodies block FcRn interaction with circulating IgG molecules as described in the Examples herein.

### Pharmaceutical Compositions

FcRn antagonists, such as FcRn antibodies or FcRn-binding fragments thereof, for use in the present invention may be provided as a pharmaceutical or diagnostic composition comprising an antagonist molecule of the present disclosure in combination with one or more of a pharmaceutically acceptable excipient, diluent or carrier. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The present disclosure also provides a process for preparation of a pharmaceutical or diagnostic composition comprising adding and mixing the antibody molecule of the present invention together with one or more of a pharmaceutically acceptable excipient, diluent or carrier.

The FcRn antagonist, such as FcRn antibodies or FcRn-binding fragments thereof may be the sole active ingredient in the pharmaceutical or diagnostic composition or may be accompanied by other active ingredients including other antibody ingredients or non-antibody ingredients such as steroids or other drug molecules, in one example these are drug molecules whose half-life is independent of FcRn binding.

Pharmaceutical compositions may be conveniently presented in dose forms containing a predetermined amount of an active agent of the invention per dose.

Therapeutic doses of the antibodies according to the present disclosure show no apparent toxicology effects in vivo.

The pharmaceutically acceptable carrier should not itself induce the production of antibodies harmful to the individual receiving the composition and should not be toxic.

Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents.

Alternatively, the FcRn antagonist, such as an FcRn antibody or FcRn-binding fragment thereof, may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the antibody or fragment, for example if the pi of the protein is in the range 8-9 or above then a formulation pH of 7 may be appropriate.

Whilst not wishing to be bound by theory it is thought that this may ultimately provide a final formulation with improved stability, for example the antibody or fragment remains in solution.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. Dosage treatment may be a single dose schedule or a multiple dose schedule. Preferably the delivery is subcutaneous. In one example the delivery is by subcutaneous infusion. In one example the delivery is not intravenous.

It will be appreciated that the active ingredient in the composition may be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, NJ. 1991).

The FcRn antagonist of the invention can be delivered dispersed in a solvent, e.g., in the form of a solution or a suspension. It can be suspended in an appropriate physiological solution, e.g., saline or other pharmacologically acceptable solvent or a buffered solution. A suspension can employ, for example, lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the FcRn antagonist in the sterile buffered solvent solution, and dispensing of the formulation into sterile receptacles by methods familiar to those of ordinary skill in the art.

Once formulated, the compositions of the disclosure can be administered directly to the human subject, although the disclosure also contemplates that the method may be employed on non-human subjects.

### Therapeutically Effective Amount

The composition preferably comprises a therapeutically effective amount of the FcRn antagonist, such as an FcRn antibody (or antigen binding fragment thereof). The term “therapeutically effective amount” as used herein refers to an amount of a therapeutic FcRn antagonist needed to treat, ameliorate or prevent a targeted disease or condition, or to exhibit a detectable therapeutic or preventative effect.

### Combination Therapies

Compositions may be administered individually to a patient or may be administered in combination (e.g. simultaneously, sequentially or separately) with other agents, drugs or hormones. In one embodiment the FcRn antagonists, such as FcRn antibodies or FcRn-binding fragments thereof according to the present disclosure are employed with a second agent. In some embodiments, the second agent is an analgesic, an antidepressant, a dopamine agonist such as pramipexole, an anti-seizure drug, an antihistamine, a hypnotic, a muscle relaxant, and an antipsychotic.

In some embodiments, the analgesic is selected from the group consisting of paracetamol (acetaminophen), ibuprofen, and opiates such as codeine, tramadol, morphine and oxycodone.

In some embodiments, the antidepressant is selected from the group consisting of a tricyclic antidepressant, a serotonin-noradrenaline reuptake inhibitor (SNRI) and a selective serotonin reuptake inhibitor (SSRI).

In some embodiments, the tricyclic antidepressant is selected from the group consisting of amitriptyline, desipramine, clomipramine, protriptyline, doxepin, imipramine, amoxapine, trazodone, nortriptyline, and trimipramine.

In some embodiments, the SNRI is selected from the group consisting of venlafaxine, duloxetine, desvenlafaxine, levomilnacipran, and milnacipran.

In some embodiments, the SSRI is selected from the group consisting of citalopram, escitalopram, fluoxetine, fluvoxamine, paroxetine, and sertraline.

In some embodiments, the anti-seizure drug is selected from the group consisting of gabapentin and pregabalin

In some embodiments, the hypnotic is a benzodiazepine or nonbenzodiazopine.

In some embodiments, the benzodiazepine is selected from the group consisting of lorazepam, clonazepam, diazepam and alprazolam.

In some embodiments, the nonbenzodiazepine is selected from the group consisting of zolpidem, zaleplon, and eszopiclone.

In some embodiments, the muscle relaxant is selected from the group consisting of cyclobenzaprine, flexeril, orphenadrine citrate, tizanidine, and carisoprodol.

In some embodiments, the antipsychotic is quetiapine.

### Example 1

### Introduction

The transfer of serum IgG from human patients with severe FMS to rodents produced typical features of clinical FMS in the rodents, including allodynia, thermal hypersensitivity, reduced grip strength, and small fiber pathology when compared with the transfer of serum IgG from healthy volunteers (Goebel et al., 2021). Fibromyalgia patients with high levels of anti-satellite glia cell IgG antibodies present with more severe symptoms (Krock et al., 2022).

The following experiments were conducted to assess the efficacy of an anti-mouse FcRn antibody (UCB4470) in vivo in an FMS patient IgG transfer model as detailed below.

### Method

Mice were administered either healthy control (HC) IgG (pooled from 4 HC donors) or

FMS patient IgG. The FMS patient IgG consisted of two pools (FMA and FMB) which were derived from patients with high pain intensity (FIQ>65 and average pain on a visual analogue scale (VAS)>60). Each pool consisted of IgG from 4 FMS patients.

All IgG pools were administered interperitoneally (i.p) at 8 mg on 2 consecutive days (day 0 & 1) to n=6 mice per group. Groups are outlined below.

Mice were treated with either an anti-murine FcRn antibody (antibody UCB4470, Smith et al. 2019) or an isotype control antibody (UCB101.4) at 30 mg/kg starting 1 day prior (Day-1) to the administration of either HC or FMS IgG pools (FMA and FMB) via an intravenous injection. UCB4470 and UCB101.4 were then administered i.p on day 2, 6, 10 and 14.

### In Vivo Groups

Group 1—8 mg IgG HC pool+isotype control antibody, UCB101.4 30 mg/kg (n=6 mice)

Group 2—8 mg IgG FMS pool A (FMA, n=6 mice) or FMS pool B (FMB, n=6 mice)+isotype control antibody, UCB101.4 30 mg/kg.

Group 3—8 mg IgG FMS pool A (FMA, n=6 mice) or FMS pool B (FMB, n=6 mice)+antibody UCB4470 30 mg/kg.

### Behavioural Assessment

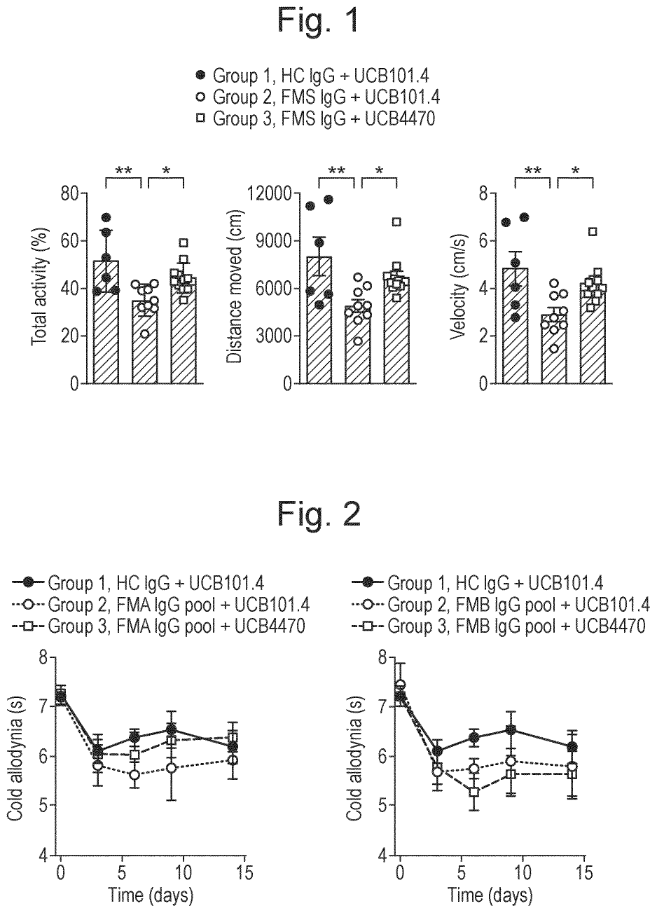

Mice in each group were assessed for behavioural changes after administration of either HC or FMS IgG. Changes in activity and movement were assessed on day 4 using an open field test together with video tracking software Ethovision XT 17 (Noldus). Mice were placed in an open field arena and their behaviour was recorded for a period of 30 minutes. Total activity, distance moved and velocity were then calculated for each mouse (FIG. 1). Data was analysed using One-Way ANOVA to determine statistical significance, p<0.05* and p<0.01**.

Cold allodynia was assessed using the ice stick test and the latency period until the mouse withdrew its paw from the cold stimulus was recorded in seconds (FIG. 2). Mice were acclimatised to the ice stick test 2 weeks prior to the start of the study. Measurements were then taken at baseline (day 0) and on day 3, 6, 9 and 14.

Injections of UCB101.4 and UCB4470 were performed between 9-10 am, behavioural assessments between 2-5 μm.

### Results

In the open field test the administration of IgG from FMS patients to mice (FMA and FMB pools) resulted in a statistically significant decrease in total activity, distance moved and velocity of the mice as compared to mice administered IgG from healthy controls (FIG. 1). This reduction in overall activity indicates that IgG from FMS patients is having an effect in mice, making them more reluctant to move as compared to mice administered HC IgG. Treatment of mice that had received IgG from FMS patients with an anti-mouse FcRn antibody at 30 mg/kg (UCB4470) resulted in a statistically significant increase in total activity, distance moved and velocity as compared to mice that had received the isotype control antibody (UCB101.4 30 mg/kg) and IgG from FMS patients (FIG. 1). Blockade of FcRn with UCB4470 therefore restores the normal activity of mice.

In the cold allodynia test all mice showed a reduction in the latency period after treatment with antibodies (UCB101.4 or UCB4470) and administration of IgG from HC or FMS pooled samples on day 3 as compared to baseline on day 0. This reduction was similar across all groups. In mice administered IgG from FMS patients (FMA and FMB) that were treated with the isotype control antibody (UCB101.4) there was an overall reduction in the latency period from day 3 until day 9 compared to mice that received IgG from HC (FIG. 2). This suggests IgG from FMS patients renders the recipient mouse hypersensitive to the cold stimulus. Treatment with UCB4470 resulted in a trend towards inhibition of this hypersensitivity when mice received IgG from the FMA pool. However, this trend was not observed with UCB4470 when mice received IgG from the FMB pool.

### Conclusion

Overall, these data indicate that the transfer of IgG from FMS patients results in behavioural differences and hypersensitivity to cold stimulus in recipient mice compared to transfer of IgG from HC. The data also indicates that treatment with an anti-FcRn antibody can restore the normal activity of mice and has a trend towards protecting against hypersensitivity to a cold stimulus in one of the two pools of IgG from FMS patients tested. The reduction of IgG by blocking FcRn may therefore have a therapeutic effect in FMS patients with high pain intensity.

### Example 2

We are conducting a Phase 2, multi-center, randomized, double-blind, placebo-controlled, proof-of-concept study to evaluate the efficacy, safety, PK, and PD of rozanolixizumab for the treatment of severe FMS. Rozanolixizumab is an anti-FcRn antibody that inhibits the binding of IgG to FcRn. The primary endpoint is the average BPI-SF interference score after 12 weeks of double-blind treatment.

The study consists of a Screening Period of up to 28 days, a 2-week, single-blind Run-in Period, 2 subsequent 12-week, double-blind Treatment Periods, followed by a 2-week, single-blind Run-out Period, and a 5-week Safety Follow-Up (SFU) Period.

Study participants are selected based on a prior confirmed diagnosis of fibromyalgia as defined by the 2016 Revisions to the 2010/2011 fibromyalgia diagnostic criteria (American College of Rheumatology Preliminary Diagnostic Criteria) plus the following characteristics during the Screening Period:
a. BPI-SF interference ≥6;
b. Study participant has been diagnosed with FMS for at least 6 months;
c. Study participant has been having FMS symptomatology for at least 2 years before enrolment.

Prior to randomisation, the participant must have confirmed:
a. Mean daily average 24 h pain intensity ≥6 and <10 assessed by Pain NRS. This will be assessed over a 10 day period within the Screening Period. Study participants require a minimum of 7 out of 10 assessments over this 10-day period within the Screening Period.
b. Pain NRS scores should be ≥4 at all completed assessments within this 10-day period.

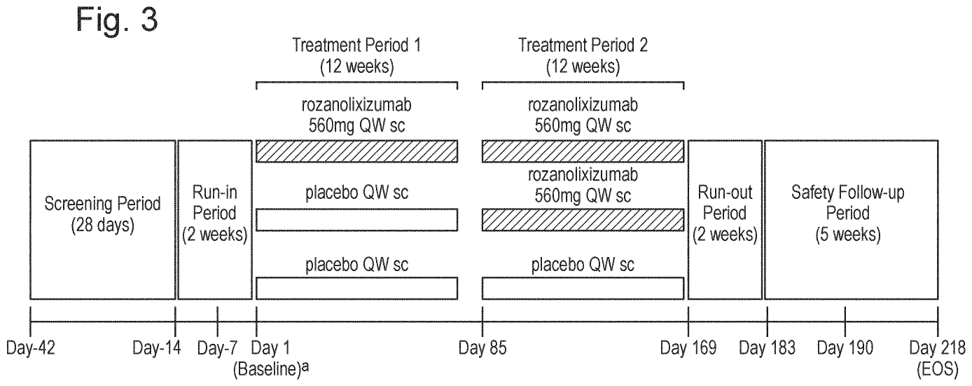

Study participants are randomized in a 1:1:1 ratio to 1 of 3 sequences. The study participants in each sequence receive the following treatment regimen during the double-blind Treatment Periods (see FIG. 3):
Sequence 1: Rozanolixizumab 560 mg subcutaneously (sc) once weekly (QW) for 24 weeks (12+12 weeks Treatment Period) (N=20, Group 1)·
Sequence 2: Placebo sc QW for 12 weeks, followed by rozanolixizumab 560 mg sc QW for 12 weeks (N=20, Group 2)·
Sequence 3: Placebo sc QW for 24 weeks (N=20, Group 3)

The initial 2-week Run-in Period and the final 2-week Run-out Period are study participant-blind, with study participants not informed that they are receiving placebo, to limit the impact of any start-of-study or end-of-study induced placebo effects. The two 12-week Treatment Periods are study participant-, investigator-, and sponsor-blind. It is important that study participants do not know when their treatment changes, whether it is going from Run-in to Period 1, Period 1 to Period 2, or Period 2 to Run-out.

After approximatively 30 study participants have been randomized and these study participants have completed their 2 double-blind Treatment Periods, an interim analysis may be conducted with the possibility of stopping for efficacy or futility.

A Safety Monitoring Committee (SMC) will regularly review the available blinded safety data (approximately every 3 months, with the option to adapt the frequency based on recruitment rates).

Results will be assessed using the Brief Pain Inventory-Short Form (BPI-SF), Pain numeric rating scale (Pain NRS), Revised Fibromyalgia Impact Questionnaire (FIQR), and the Fatigue numeric rating scale (Fatigue NRS).

### Brief Pain Inventory-Short Form (BPI-SF)

The BPI-SF was designed to measure multiple clinically relevant aspects of pain such as pain intensity and interference from pain in cancer populations. There are two versions; the short version is the most commonly used and is often included in the context of clinical trials (Williams and Arnold, 2011). The short form of the BPI is a self-administered questionnaire used to evaluate the severity of a study participant's pain and the impact of this pain on the study participant's daily functioning. The BPI-SF assesses for the location of pain, pain intensity, and functional interference from pain. The BPI-SF should be completed by the study participants in a quiet place prior to other patient-reported outcomes (PROs) or protocol-specified assessments at each visit including on dosing days.

The 4 BPI-SF severity items include: worst pain in last 24 hours, least pain in last 24 hours, pain on average, and pain right now. Each item is rated on a 0 (no pain) to 10 (pain as bad as you can imagine) scale with a recall period of 24 hours.

The 7 BPI-SF interference items include: general activity, mood, walking ability, normal work (including housework), relations with other people, sleep, and enjoyment of life. Each item is rated on a 0 (does not interfere) to 10 (completely interferes) scale with a recall period of 24 hours. The arithmetic mean of the 7 interference items can be used as a measure of pain interference.

### Pain Numeric Rate Scale (Pain NRS)

The Pain NRS is a numeric version of the VAS (visual analog scale) in which a respondent selects a whole number that best describes “How much pain have you experienced on average over the past 24 hours?” The 11-point Pain NRS ranges from 0 (no pain) to 10 (pain as bad as you can imagine).

The daily average pain over the previous 24 hours is captured using the Pain NRS through a diary over a 10-day period within the Screening Period. Study participants require a minimum of 7 out of the 10 daily Pain NRS assessments over this 10-day period to be eligible for randomization. All recorded Pain NRS scores over the 10-day period within the Screening Period are used to calculate the mean daily average 24 hours pain intensity ≥6 and <10 for inclusion (for example, if 10 daily scores are available take the mean of the 10 days; if 9 daily scores are available take the mean of the 9 days and so on).

During the Run-in Period and at the end of the Treatment Periods, the pain NRS is collected daily for 7 consecutive days following the assessment visit. For all other visits, the average pain for the preceding 24 hours prior to the visit is captured by the pain NRS for that day only.

### Revised Fibromyalgia Impact Questionnaire (FIQR)

The FIQR is a 21-item questionnaire with a recall period of 7 days. The FIQR includes 3 domains: function, overall impact, and symptoms. Each item is based on an 11-point numeric rating scale. The FIQR is completed by the participant during study visits.

### Fatigue Numeric Rating Scale (Fatigue NRS)

The Fatigue NRS is a numeric version of the VAS in which a respondent selects a whole number that best describes “How much fatigue have you experienced on average over the past 24 hours?” The 11-point Fatigue NRS ranges from 0 (no fatigue) to 10 (fatigue as bad as you can imagine). The Fatigue NRS will be completed by the participant at home/during study visits.

Sequences

| SEQ ID | | |
|---|---|---|
| NO | Description | Sequence |
| 1 | Rozanolixizumab heavy | EVPLVESGGGLVQPGGSLRLSCAVS |
| | chain variable region | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVS |
| 2 | Rozanolixizumab light | DIQMTQSPSSLSASVGDRVTITCKSS |
| | chain variable region | QSLVGASGKTYLYWLFQKPGKAPK |
| | | RLIYLVSTLDSGIPSRFSGSGSGTEFT |
| | | LTISSLQPEDFATYYCLQGTHFPHTF |
| | | GQGTKLEIK |
| 3 | Human heavy chain JH4 | EVQLVESGGGLVQPGGSLRLSCAAS |
| | acceptor framework | GFTFSSYWMSWVRQAPGKGLEWVA |
| | | NIKQDGSEKYYVDSVKGRFTISRDN |
| | | AKNSLYLQMNSLRAEDTAVYYCAR |
| | | YFDYWGQGTLVTVS |
| 4 | HCDR1 | GFTFSNYGMV |
| 5 | HCDR2 | YIDSDGDNTYYRDSVKG |
| 6 | HCDR3 | GIVRPFLY |
| 7 | Human JH4 | YFDYWGQGTLVTVS |
| 8 | Humanised light chain | DIQMTQSPSSLSASVGDRVTITCRAS |
| | framework | QGIRNDLGWYQQKPGKAPKRLIYA |
| | | ASSLQSGVPSRFSGSGSGTEFTLTISS |
| | | LQPEDFATYYCLQHNSYPYTFGQGT |
| | | KLEIK |
| 9 | LCDR1 | KSSQSLVGASGKTYLY |
| 10 | LCDR2 | LVSTLDS |
| 11 | LCDR3 | LQGTHFPHT |
| 12 | JK2 sequence | YTFGQGTKLEIK |
| 13 | Rozanolixizumab light | DIQMTQSPSSLSASVGDRVTITCKSS |
| | chain | QSLVGASGKTYLYWLFQKPGKAPK |
| | | RLIYLVSTLDSGIPSRFSGSGSGTEFT |
| | | LTISSLQPEDFATYYCLQGTHFPHTF |
| | | GQGTKLEIKRTVAAPSVFIFPPSDEQ |
| | | LKSGTASVVCLLNNFYPREAKVQW |
| | | KVDNALQSGNSQESVTEQDSKDSTY |
| | | SLSSTLTLSKADYEKHKVYACEVTH |
| | | QGLSSPVTKSFNRGEC |
| 14 | Fab heavy chain | EVPLVESGGGLVQPGGSLRLSCAVS |
| | | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVSSASTKG |
| | | PSVFPLAPSSKSTSGGTAALGCLVKD |
| | | YFPEPVTVSWNSGALTSGVHTFPAV |
| | | LQSSGLYSLSSVVTVPSSSLGTQTYIC |
| | | NVNHKPSNTKVDKKVEPKSCDKTH |
| | | TC |
| 15 | IgG1 heavy chain | EVPLVESGGGLVQPGGSLRLSCAVS |
| | | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVSSASTKG |
| | | PSVFPLAPSSKSTSGGTAALGCLVKD |
| | | YFPEPVTVSWNSGALTSGVHTFPAV |
| | | LQSSGLYSLSSVVTVPSSSLGTQTYIC |
| | | NVNHKPSNTKVDKKVEPKSCDKTH |
| | | TCPPCPAPELLGGPSVFLFPPKPKDTL |
| | | MISRTPEVTCVVVDVSHEDPEVKFN |
| | | WYVDGVEVHNAKTKPREEQYNSTY |
| | | RVVSVLTVLHQDWLNGKEYKCKVS |
| | | NKALPAPIEKTISKAKGQPREPQVYT |
| | | LPPSRDELTKNQVSLTCLVKGFYPSD |
| | | IAVEWESNGQPENNYKTTPPVLDSD |
| | | GSFFLYSKLTVDKSRWQQGNVFSCS |
| | | VMHEALHNHYTQKSLSLSPGK |
| 16 | IgG4 heavy chain | EVPLVESGGGLVQPGGSLRLSCAVS |
| | (no P change) | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVSSASTKG |
| | | PSVFPLAPCSRSTSESTAALGCLVKD |
| | | YFPEPVTVSWNSGALTSGVHTFPAV |
| | | LQSSGLYSLSSVVTVPSSSLGTKTYT |
| | | CNVDHKPSNTKVDKRVESKYGPPCP |
| | | SCPAPEFLGGPSVFLFPPKPKDTLMIS |
| | | RTPEVTCVVVDVSQEDPEVQFNWY |
| | | VDGVEVHNAKTKPREEQFNSTYRV |
| | | VSVLTVLHQDWLNGKEYKCKVSNK |
| | | GLPSSIEKTISKAKGQPREPQVYTLPP |
| | | SQEEMTKNQVSLTCLVKGFYPSDIA |
| | | VEWESNGQPENNYKTTPPVLDSDGS |
| | | FFLYSRLTVDKSRWQEGNVFSCSVM |
| | | HEALHNHYTQKSLSLSLGK |
| 17 | Rozanolixizumab heavy | EVPLVESGGGLVQPGGSLRLSCAVS |
| | chain | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVSSASTKG |
| | | PSVFPLAPCSRSTSESTAALGCLVKD |
| | | YFPEPVTVSWNSGALTSGVHTFPAV |
| | | LQSSGLYSLSSVVTVPSSSLGTKTYT |
| | | CNVDHKPSNTKVDKRVESKYGPPCP |
| | | PCPAPEFLGGPSVFLFPPKPKDTLMIS |
| | | RTPEVTCVVVDVSQEDPEVQFNWY |
| | | VDGVEVHNAKTKPREEQFNSTYRV |
| | | VSVLTVLHQDWLNGKEYKCKVSNK |
| | | GLPSSIEKTISKAKGQPREPQVYTLPP |
| | | SQEEMTKNQVSLTCLVKGFYPSDIA |
| | | VEWESNGQPENNYKTTPPVLDSDGS |
| | | FFLYSRLTVDKSRWQEGNVFSCSVM |
| | | HEALHNHYTQKSLSLSLGK |
| 18 | Albumin binding heavy | EVPLVESGGGLVQPGGSLRLSCAVS |
| | chain | GFTFSNYGMVWVRQAPGKGLEWV |
| | | AYIDSDGDNTYYRDSVKGRFTISRD |
| | | NAKSSLYLQMNSLRAEDTAVYYCT |
| | | TGIVRPFLYWGQGTLVTVSSASTKG |
| | | PSVFPLAPSSKSTSGGTAALGCLVKD |
| | | YFPEPVTVSWNSGALTSGVHTFPAV |
| | | LQSSGLYSLSSVVTVPSSSLGTQTYIC |
| | | NVNHKPSNTKVDKKVEPKSCSGGG |
| | | GSGGGGTGGGGSEVQLLESGGGLV |
| | | QPGGSLRLSCAVSGIDLSNYAINWV |
| | | RQAPGKCLEWIGIIWASGTTFYATW |
| | | AKGRFTISRDNSKNTVYLQMNSLRA |
| | | EDTAVYYCARTVPGYSTAPYFDLW |
| | | GQGTLVTVSS |
| 19 | Albumin binding light | DIQMTQSPSSLSASVGDRVTITCKSS |
| | chain 1 | QSLVGASGKTYLYWLFQKPGKAPK |
| | | RLIYLVSTLDSGIPSRFSGSGSGTEFT |
| | | LTISSLQPEDFATYYCLQGTHFPHTF |
| | | GQGTKLEIKRTVAAPSVFIFPPSDEQ |
| | | LKSGTASVVCLLNNFYPREAKVQW |
| | | KVDNALQSGNSQESVTEQDSKDSTY |
| | | SLSSTLTLSKADYEKHKVYACEVTH |
| | | QGLSSPVTKSFNRGECSGGGGSGGG |
| | | GSGGGGSDIQMTQSPSSVSASVGDR |
| | | VTITCQSSPSVWSNFLSWYQQKPGK |
| | | APKLLIYEASKLTSGVPSRFSGSGSG |
| | | TDFTLTISSLQPEDFATYYCGGGYSSI |
| | | SDTTFGCGTKVEIKRT |
| 20 | Albumin binding light | DIQMTQSPSSLSASVGDRVTITCKSS |
| | chain 2 | QSLVGASGKTYLYWLFQKPGKAPK |
| | | RLIYLVSTLDSGIPSRFSGSGSGTEFT |
| | | LTISSLQPEDFATYYCLQGTHFPHTF |
| | | GQGTKLEIKRTVAAPSVFIFPPSDEQ |
| | | LKSGTASVVCLLNNFYPREAKVQW |
| | | KVDNALQSGNSQESVTEQDSKDSTY |
| | | SLSSTLTLSKADYEKHKVYACEVTH |
| | | QGLSSPVTKSFNRGECGGGGSGGGG |
| | | SGGGGSDIQMTQSPSSVSASVGDRV |
| | | TITCQSSPSVWSNFLSWYQQKPGKA |
| | | PKLLIYEASKLTSGVPSRFSGSGSGT |
| | | DFTLTISSLQPEDFATYYCGGGYSSIS |
| | | DTTFGCGTKVEIKRT |
| 21 | Human FcRn alpha chain | AESHLSLLYHLTAVSSPAPGTPAFW |
| | extracellular sequence | VSGWLGPQQYLSYNSLRGEAEPCGA |
| | | WVWENQVSWYWEKETTDLRIKEKL |
| | | FLEAFKALGGKGPYTLQGLLGCELG |
| | | PDNTSVPTAKFALNGEEFMNFDLKQ |
| | | GTWGGDWPEALAISQRWQQQDKA |
| | | ANKELTFLLFSCPHRLREHLERGRG |
| | | NLEWKEPPSMRLKARPSSPGFSVLT |
| | | CSAFSFYPPELQLRFLRNGLAAGTGQ |
| | | GDFGPNSDGSFHASSSLTVKSGDEH |
| | | HYCCIVQHAGLAQPLRVELESPAKS |
| | | S |
| 22 | β2 microglobulin | IQKTPQIQVYSRHPPENGKPNFLNCY |
| | | VSQFHPPQIEIELLKNGKKIPNIEMSD |
| | | LSFSKDWSFYILAHTEFTPTETDVYA |
| | | CRVKHVILKEPKTVTWDRDM |

### REFERENCES

Ahimsadasan N, Reddy V, Kumar A. Neuroanatomy, Dorsal Root Ganglion. [Updated 2021 Sep. 7]
Adair, J. R., & Lawson, A. D. G. (2005 May). Therapeutic antibodies. Drug Design Reviews Online. https://doi.org/10.2174/1567269053828800
Altschul S F, Gish W, Miller W, Myers E W, Lipman D J. Basic local alignment search tool. J Mol Biol. 1990 Oct. 5; 215 (3): 403-10. doi: 10.1016/S0022-2836 (05) 80360-2. PMID: 2231712.
Altschul S F, Madden T L, Schäffer A A, Zhang J, Zhang Z, Miller W, Lipman D J. Gapped BLAST and PSI-BLAST: a new generation of protein database search programs. Nucleic Acids Res. 1997 Sep. 1; 25 (17): 3389-402. doi: 10.1093/nar/25.17.3389. PMID: 9254694; PMCID: PMC146917.
Anderson, C. L. et al. Perspective-FcRn transports albumin: relevance to immunology and medicine. Trends Immunol. 27, 343-348 (2006).
Angal et al., A single amino acid substitution abolishes the heterogeneity of chimeric mouse/human (IgG4) antibody, Molecular Immunology, 1993, 30 (1), 105-108
Babcook, J. et al., A novel strategy for generating monoclonal antibodies from single, isolated lymphocytes producing antibodies of defined specificities. 1996, Proc. Natl. Acad. Sci. USA 93 (15): 7843-7848
Chandran A, Schaefer C, Ryan K, et al. The comparative economic burden of mild, moderate, and severe fibromyalgia: results from a retrospective chart review and cross-sectional survey of working-age U.S. adults. J Manag Care Pharm 2012; 18:415-26.
Chothia C, Lesk A M. Canonical structures for the hypervariable regions of immunoglobulins. J Mol Biol. 1987 Aug. 20; 196 (4): 901-17. doi: 10.1016/0022-2836 (87) 90412-8. PMID: 3681981.
Claypool et al., 2002, Functional reconstitution of human FcRn in Madin-Darby canine kidney cells requires co-expressed human beta 2-microglobulin. Journal of Biological Chemistry, 277, 31, 28038-28050
Cole et al., Monoclonal Antibodies and Cancer Therapy, pp77-96, Alan R Liss, Inc., 1985
Computational Molecular Biology, Lesk, A. M., ed., Oxford University Press, New York, 1988 Biocomputing. Informatics and Genome Projects, Smith, D. W., ed., Academic Press, New York, 1993
Computer Analysis of Sequence Data, Part 1, Griffin, A. M., and Griffin, H. G., eds., Humana Press, New Jersey, 1994
Crameri, A., Raillard, S A., Bermudez, E. et al. DNA shuffling of a family of genes from diverse species accelerates directed evolution. Nature 391, 288-291 (1998). https://doi.org/10.1038/34663
Firan M, Bawdon R, Radu C et al (2001) The MHC class I related receptor, FcRn, plays an essential role in the maternofetal transfer of gammaglobulin in humans. Int Immunol 13:993-1002
Gable K L and Guptill J T (2020) Antagonism of the Neonatal Fc Receptor as an Emerging Treatment for Myasthenia Gravis. Front. Immunol. 10:3052. doi: 10.3389/fimmu.2019.03052
Gish, W., States, D. Identification of protein coding regions by database similarity search. Nat Genet 3, 266-272 (1993). https://doi.org/10.1038/ng0393-266
Goebel A, Krock E, Gentry C, Israel M R, Jurczak A, Urbina C M, Sandor K, Vastani N, Maurer M, Cuhadar U, Sensi S, Nomura Y, Menezes J, Baharpoor A, Brieskorn L, Sandström A, Tour J, Kadetoff D, Haglund L, Kosek E, Bevan S, Svensson C I, Andersson D A. Passive transfer of fibromyalgia symptoms from patients to mice. J Clin Invest. 2021 Jul. 1; 131 (13): e144201. doi: 10.1172/JCI144201. PMID: 34196305; PMCID: PMC8245181.
R. J. Harris, “Processing of C-Terminal Lysine and Arginine Residues of Proteins Isolated from Mammalian Cell Culture,” Journal of Chromatography A, Vol. 705, No. 1, 1995, pp. 129-134. http://dx.doi.org/10.1023/A: 1015929109894
Häuser W, Ablin J, Fitzcharles M A, et al. Fibromyalgia. Nat Rev Dis Primers. 2015; 1:15022. doi: 10.1038/nrdp.2015.22. PMID: 27189527.
Hieter P A, Maizel J V Jr, Leder P. Evolution of human immunoglobulin kappa J region genes. J Biol Chem. 1982 Feb. 10; 257 (3): 1516-22. PMID: 6276389.
Holliger, P., Hudson, P. Engineered antibody fragments and the rise of single domains. Nat Biotechnol 23, 1126-1136 (2005). https://doi.org/10.1038/nbt1142
Kabat et al., 1987, in Sequences of Proteins of Immunological Interest, U S Department of Health and Human Services, NIH, USA
Kashmiri S V, De Pascalis R, Gonzales N R, Schlom J. SDR grafting—a new approach to antibody humanization. Methods. 2005 May; 36 (1): 25-34. doi: 10.1016/j.ymeth.2005.01.003. PMID: 15848072.
KÖHLER, G., MILSTEIN, C. Continuous cultures of fused cells secreting antibody of predefined specificity. Nature 256, 495-497 (1975). https://doi.org/10.1038/256495a0
Kozbor et al., The production of monoclonal antibodies from human lymphocytes1983, Immunology Today, 4:72
Krock et al., 2022, Fibromyalgia patients with high levels of anti-satellite glia cell IgG antibodies present with more severe symptoms, bioRxiv preprint doi: https://doi.org/10.1101/2022.07.06.498940
Low, N. M., Holliger, P. and Winter, G. (1996) Mimicking Somatic Hypermutation: Affinity Maturation of Antibodies Displayed on Bacteriophage Using Bacterial Mutator Strain. Journal of Molecular Biology, 260, 359-368.
Madden, T. L., R. L. Tatusov, and J. Zhang. 1996. Application of network BLAST server. Methods Enzymol. 266:131-141.
Marks, J., Griffiths, A., Malmqvist, M. et al. By-Passing Immunization: Building High Affinity Human Antibodies by Chain Shuffling. Nat Biotechnol 10, 779-783 (1992). https://doi.org/10.1038/nbt0792-779
Mezo A R, McDonnell K A, Hehir C A, Low S C, Palombella V J, Stattel J M, Kamphaus G D, Fraley C, Zhang Y, Dumont J A, Bitonti A J. Reduction of IgG in nonhuman primates by a peptide antagonist of the neonatal Fc receptor FcRn. Proc Natl Acad Sci USA. 2008 Feb. 19; 105 (7): 2337-42. doi: 10.1073/pnas.0708960105. Epub 2008 Feb. 12. PMID: 18272495; PMCID: PMC2268137.
Raimund J. Ober, Caius G. Radu, Victor Ghetie, E. Sally Ward, Differences in promiscuity for antibody-FcRn interactions across species: implications for therapeutic antibodies, International Immunology, Volume 13, Issue 12, December 2001, Pages 1551-1559, https://doi.org/10.1093/intimm/13.12.1551
Patten et al., Applications of DNA shuffling to pharmaceuticals and vaccines, Curr. Opin. Biotechnol., 8, 724-733, 1997
Peter et al. Targeting FcRn for immunomodulation: Benefits, risks, and practical considerations. J Allergy Clin Immunol. 2020 September; 146 (3): 479-491.e5. doi: 10.1016/j.jaci.2020.07.016. PMID: 32896308; PMCID: PMC7471860.
Riechmann, L., Clark, M., Waldmann, H. et al. Reshaping human antibodies for therapy. Nature 332, 323-327 (1988). https://doi.org/10.1038/332323a0
Scatchard et al., The Attractions of Proteins for Small Molecules and Ions, 1949 (Ann. K Y. Acad. Sci. 51:660-672)
Sequence Analysis in Molecular Biology, von Heinje, G., Academic Press, 1987
Sequence Analysis Primer, Gribskov, M. and Devereux, J., eds., M Stockton Press, New York, 1991
Smith et al., Generation of two high affinity anti-mouse FcRn antibodies: Inhibition of IgG recycling in wild type mice and effect in a mouse model of immune thrombocytopenia. International Immunopharmacology, 55, 362-365, 2019
Thompson et al., Affinity Maturation of a High-affinity Human Monoclonal Antibody Against the Third Hypervariable Loop of Human Immunodeficiency Virus: Use of Phage Display to Improve Affinity and Broaden Strain Reactivity J. Mol. Biol., 256, 77-88, 1996
Vaughan, T., Osbourn, J. & Tempest, P. Human antibodies by design. Nat Biotechnol 16, 535-539 (1998). https://doi.org/10.1038/nbt0698-535
Verma et al., Antibody engineering: Comparison of bacterial, yeast, insect and mammalian expression systems, 1998, Journal of Immunological Methods, 216, 165-181
White L A, Birnbaum H G, Kaltenboeck A, et al. Employees with fibromyalgia: medical comorbidity, healthcare costs, and work loss. J Occup Environ Med 2008; 50 (1): 13-24.
Wolff et al, Monoclonal Antibody Homodimers: Enhanced Antitumor Activity in Nude Mice, Cancer Res. 53:2560-65 (1993))
Yang et al., CDR Walking Mutagenesis for the Affinity Maturationof a Potent Human
Anti-HIV-1 Antibody into the Picomolar Range, J. Mol. Biol., 254, 392-403, 1995 Zhang J, Madden T L. PowerBLAST: a new network BLAST application for interactive or automated sequence analysis and annotation. Genome Res. 1997 June; 7 (6): 649-56. doi: 10.1101/gr.7.6.649. PMID: 9199938; PMCID: PMC310664.

### ASPECTS OF THE DISCLOSURE

1. A method of treating or preventing fibromyalgia syndrome (FMS) in an individual, the method comprising administering to the individual a therapeutically effective amount of an FcRn antagonist, wherein the FcRn antagonist inhibits binding of IgG to FcRn receptor.
2. The method of aspect 1, wherein the FMS is severe FMS.
3. The method of aspect 1 or 2, wherein the individual has a pain intensity of greater than 5 and less than 10 (i.e. equal to or greater than 6 and less than 10) on the pain numeric rating scale (pain NRS).
4. The method of any one of aspects 1-3, wherein the individual has a brief pain inventory-short form (BPI-SF) interference score of greater than or equal to 6.
5. The method of any one of aspects 1-4, wherein the individual has a Fibromyalgia Impact Questionnaire (FIQR) score of greater than or equal to 64.
6. The method of any one of aspects 1-5, wherein the individual has a fatigue numeric rating scale (fatigue NRS) score of greater than or equal to 5.
7. The method of any one of aspects 1-6, wherein the individual has a pain intensity of greater than 5 and less than 10 (i.e. equal to or greater than 6 and less than 10) on the pain numeric rating scale (pain NRS), a brief pain inventory-short form (BPI-SF) interference score of greater than or equal to 6, a Fibromyalgia Impact Questionnaire (FIQR) score of greater than or equal to 64, and a fatigue numeric rating scale (fatigue NRS) score of greater than or equal to 5.
8. The method of any one of aspects 1-7, wherein treating FMS comprises reducing or eliminating one or more of the symptoms of FMS.
9. The method of aspect 8, wherein the one or more symptoms of FMS are selected from the group consisting of pain, aching, burning sensation, stabbing pain, hyperalgesia, allodynia, stiffness, muscle spasm, fatigue, reduced sleep quality, impaired memory, attention deficit, concentration deficit, impaired speech, headache, migraine, constipation, diarrhoea, dizziness, clumsiness, anxiety, and depression.
10. The method of any one of aspect 1-9, wherein the method reduces the brief pain inventory-short form (BPI-SF) average interference score of the individual.
11. The method of aspect 10, wherein the method reduces the BPI-SF score of the individual by at least 1, at least 2, at least 3, at least 4, at least 5, at least 6, at least 7, at least 8, or at least 9.
12. The method of any one of aspects 1-11, wherein the method reduces the Fibromyalgia Impact Questionnaire (FIQR) score of the individual.
13. The method of aspect 12, wherein the method reduces the FIQR score of the individual by at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, or at least 95.
14. The method of any one of aspects 1-13, wherein the method reduces the average daily pain score of the individual.
15. The method of aspect 14, wherein the average daily pain score is measured using the pain numeric rating scale (NRS).
16. The method of aspect 15, wherein the method reduces the average daily pain score of the individual by at least 1, at least 2, at least 3, at least 4, at least 5, at least 6, at least 7, at least 8, or at least 9.
17. The method of any one of aspects 1-16, wherein the method reduces the fatigue score of the individual.
18. The method of aspect 17, wherein the fatigue score is measured using the fatigue numeric rating scale (NRS).
19. The method of aspect 18, wherein the method reduces the fatigue score by at least 1, at least 2, at least 3, at least 4, at least 5, at least 6, at least 7, at least 8, or at least 9.
20. The method of any one of aspects 1-19, wherein administering the FcRn antagonist results in a reduction in the individual of serum IgG of at least 10%, at least 20%, at least 30%, at least 40%, at least 50%, at least 60%, at least 70%, at least 80%, or at least 90%.
21. The method of any one of aspects 1-20, wherein administering the FcRn antagonist results in a reduction in serum IgG levels of from 60 to 80%.
22. The method of any one of aspects 1-21, wherein administering the FcRn antagonist results in a reduction in serum IgG levels of about 70%.
23. The method of any one of aspects 1-22, wherein serum from the individual produces a higher percentage of IgG-bound satellite glial cells (SGCs) or an increased SGC-binding intensity compared to serum from a healthy control patient.
24. The method of any one of aspects 1-23, wherein administration of the FcRn antagonist results in a reduction of at least 10%, at least 20%, at least 30%, at least 40%, at least 50%, at least 60%, at least 70%, at least 80%, or at least 90% in the level of anti-SGC IgG in the individual.
25. The method of any one of aspects 1-24, wherein the FcRn antagonist is an Fc-fragment which binds to the FcRn receptor.
26. The method of aspect 25, wherein the Fc-fragment is efgartigimod.
27. The method of any one of aspects 1-26, wherein the FcRn antagonist is a peptide that blocks binding of IgG to the FcRn receptor.
28. The method of aspect 27, wherein the peptide is SYN1436.
29. The method of any one of aspects 1-24, wherein the FcRn antagonist is an anti-FcRn antibody or FcRn-binding fragment thereof.
30. The method of aspect 29, wherein the anti-FcRn antibody or FcRn-binding fragment is selected from the list consisting of rozanolixizumab, nipocalimab, orilanolimab, CSL730/M230, ABY-039 and RVT-1401 (HL161).
31. The method of aspect 29 or aspect 30, wherein the anti-FcRn antibody or FcRn-binding fragment thereof is rozanolixizumab.
32. The method of any one of aspects 29-31, wherein the anti-FcRn antibody or FcRn-binding fragment thereof comprises:
(a) a heavy chain or heavy chain fragment having a variable region, wherein said variable region comprises three CDRs having the sequences given in SEQ ID NO: 4 for CDR H1, SEQ ID NO: 5 for CDR H2, and SEQ ID NO: 6 for CDR H3, and
(b) a light chain or light chain fragment having a variable region, wherein said variable region comprises three CDRs having the sequences given in SEQ ID NO: 9 for CDR L1, SEQ ID NO: 10 for CDR L2, and SEQ ID NO: 11 for CDR L3.
   33. The method of any one of aspects 29-32, wherein the anti-FcRn antibody or FcRn-binding fragment thereof comprises a heavy chain variable region having the sequence given in SEQ ID NO: 1, and a light chain variable region having the sequence given in SEQ ID NO: 2.
   34. The method of any one of aspects 29-33, wherein the anti-FcRn antibody or FcRn-binding fragment is a scFv, Fv, Fab or Fab′ fragment.
   35. The method of aspect 34, wherein the Fab fragment comprises a heavy chain having the sequence given in SEQ ID NO: 14 and a light chain have the sequence given in SEQ ID NO: 13.
   36. The method of any one of aspects 29-35, wherein the anti-FcRn antibody comprises a heavy chain having the sequence given in SEQ ID NO: 17 and a light chain having the sequence given in SEQ ID NO: 13.
   37. The method of any one of aspects 29-36, wherein the anti-FcRn antibody or FcRn-binding fragment thereof is administered at a dose of from 1 mg/kg to 50 mg/kg.
   38. The method of any one of aspects 29-27, wherein the anti-FcRn antibody or FcRn-binding fragment thereof is administered at a dose of from 4 mg/kg to 20 mg/kg.
   39. The method of any one of aspects 29-38, wherein the anti-FcRn antibody or FcRn-binding fragment thereof is administered at a dose of from 7 mg/kg to 10 mg/kg.
   40. The method of any one of aspects 29-39, wherein the anti-FcRn antibody or FcRn-binding fragment thereof is administered at a dose of 560 mg.
   41. The method of any one of aspects 29-40, wherein the anti-FcRn antibody or FcRn-binding fragment thereof is administered subcutaneously.
   42. The method of any one of aspects 29-41, wherein the anti-FcRn antibody or FcRn-binding fragment thereof is administered once a day, three times a week, twice a week, once a week, once every two weeks, once every three weeks, once every month, once every six weeks, once every two months, once every three months, once every four months, once every five months, or once every six months.
   43. The method of any one of aspects 29-42, wherein the anti-FcRn antibody or FcRn-binding fragment thereof is administered once a week.
   44. The method of any one of aspects 1-43, wherein the method further comprises administering a therapeutically effective amount of a second agent for treating FMS.
   45. The method of aspect 44, wherein the second agent is selected from the group consisting of an analgesic, an antidepressant, a dopamine agonist, an anti-seizure drug, an antihistamine, a hypnotic, a muscle relaxant, and an antipsychotic.
   46. The method of aspect 45, wherein the analgesic is selected from the group consisting of paracetamol (acetaminophen), ibuprofen, and opiates such as codeine, tramadol, morphine and oxycodone.
   47. The method of aspect 45, wherein the antidepressant is selected from the group consisting of a tricyclic antidepressant, a serotonin-noradrenaline reuptake inhibitor (SNRI) and a selective serotonin reuptake inhibitor (SSRI).
   48. The method of aspect 47, wherein the tricyclic antidepressant is selected from the group consisting of amitriptyline, desipramine, clomipramine, protriptyline, doxepin, imipramine, amoxapine, trazodone, nortriptyline, and trimipramine.
   49. The method of aspect 47, wherein the SNRI is selected from the group consisting of venlafaxine, duloxetine, desvenlafaxine, levomilnacipran, and milnacipran.
   50. The method of aspect 47, wherein the SSRI is selected from the group consisting of citalopram, escitalopram, fluoxetine, fluvoxamine, paroxetine, and sertraline.
   51. The method of aspect 45, wherein the anti-seizure drug is selected from the group consisting of gabapentin and pregabalin.
   52. The method of aspect 45, wherein the hypnotic is a benzodiazepine or nonbenzodiazopine.
   53. The method of aspect 52, wherein the benzodiazepine is selected from the group consisting of lorazepam, clonazepam, diazepam and alprazolam.
   54. The method of aspect 52, wherein the nonbenzodiazepine is selected from the group consisting of zolpidem, zaleplon, and eszopiclone.
   55. The method of aspect 45, wherein the muscle relaxant is selected from the group consisting of cyclobenzaprine, flexeril, orphenadrine citrate, tizanidine, and carisoprodol.
   56. The method of aspect 45, wherein the antipsychotic is quetiapine.
   57. An FcRn antagonist for use in a method of treating or preventing fibromyalgia syndrome (FMS) in an individual, wherein the FcRn antagonist inhibits binding of IgG to FcRn receptor.
   58. Use of an FcRn antagonist for the manufacture of a medicament for treating or preventing fibromyalgia syndrome (FMS) in an individual, wherein the FcRn antagonist inhibits binding of IgG to FcRn receptor.

1. A method of treating or preventing fibromyalgia syndrome (FMS) in an individual, the method comprising administering to the individual a therapeutically effective amount of an FcRn antagonist, wherein the FcRn antagonist is an anti-FcRn antibody or FcRn-binding antibody fragment comprising:
(a) a heavy chain or heavy chain fragment having a variable region, wherein said variable region comprises three CDRs having the sequences given in SEQ ID NO: 4 for CDR H1, SEQ ID NO: 5 for CDR H2, and SEQ ID NO: 6 for CDR H3, and
(b) a light chain or light chain fragment having a variable region, wherein said variable region comprises three CDRs having the sequences given in SEQ ID NO: 9 for CDR L1, SEQ ID NO: 10 for CDR L2, and SEQ ID NO: 11 for CDR L3.

2. The method of claim 1, wherein the anti-FcRn antibody or FcRn-binding fragment thereof comprises a heavy chain variable region having the sequence given in SEQ ID NO: 1, and a light chain variable region having the sequence given in SEQ ID NO: 2.

3. The method of claim 1 or claim 2, wherein the FcRn-binding fragment is a scFv, Fv, Fab or Fab′ fragment.

4. The method of claim 3, wherein the Fab fragment comprises a heavy chain having the sequence given in SEQ ID NO: 14 and a light chain have the sequence given in SEQ ID NO: 13.

5. The method of claim 1 or claim 2, wherein the anti-FcRn antibody comprises a heavy chain having the sequence given in SEQ ID NO: 17 and a light chain having the sequence given in SEQ ID NO: 13.

6. The method of claim 1, 2 or 5, wherein the anti-FcRn antibody is rozanolixizumab.

7. The method of any one of claims 1-6, wherein the FMS is severe FMS.

8. The method of any one of claims 1-7, wherein the individual has a pain intensity of greater than 5 and less than 10 on the pain numeric rating scale (pain NRS).

9. The method of any one of claims 1-7, wherein the individual has a pain intensity of equal to or greater than 6 and less than 10 on the pain numeric rating scale (pain NRS).

10. The method of any one of claims 1-9, wherein the individual has a brief pain inventory-short form (BPI-SF) interference score of greater than or equal to 6.

11. The method of any one of claims 1-10, wherein the individual has a Fibromyalgia Impact Questionnaire (FIQR) score of greater than or equal to 64.

12. The method of any one of claims 1-11, wherein the individual has a fatigue numeric rating scale (fatigue NRS) score of greater than or equal to 5.

13. The method of any one of claims 1-12, wherein the individual has a pain intensity of greater than 5 and less than 10 (i.e. equal to or greater than 6 and less than 10) on the pain numeric rating scale (pain NRS), a brief pain inventory-short form (BPI-SF) interference score of greater than or equal to 6, a Fibromyalgia Impact Questionnaire (FIQR) score of greater than or equal to 64, and a fatigue numeric rating scale (fatigue NRS) score of greater than or equal to 5.

14. The method of any one of claims 1-13, wherein treating FMS comprises reducing or eliminating one or more of the symptoms of FMS.

15. The method of claim 14, wherein the one or more symptoms of FMS are selected from the group consisting of pain, aching, burning sensation, stabbing pain, hyperalgesia, allodynia, stiffness, muscle spasm, fatigue, reduced sleep quality, impaired memory, attention deficit, concentration deficit, impaired speech, headache, migraine, constipation, diarrhoea, dizziness, clumsiness, anxiety, and depression.

16. The method of any one of claim 1-15, wherein the method reduces the brief pain inventory-short form (BPI-SF) average interference score of the individual.

17. The method of claim 16, wherein the method reduces the BPI-SF score of the individual by at least 1, at least 2, at least 3, at least 4, at least 5, at least 6, at least 7, at least 8, or at least 9.

18. The method of any one of claims 1-17, wherein the method reduces the Fibromyalgia Impact Questionnaire (FIQR) score of the individual.

19. The method of claim 18, wherein the method reduces the FIQR score of the individual by at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, or at least 95.

20. The method of any one of claims 1-19, wherein the method reduces the average daily pain score of the individual.

21. The method of claim 20, wherein the average daily pain score is measured using the pain numeric rating scale (NRS).

22. The method of claim 21, wherein the method reduces the average daily pain score of the individual by at least 1, at least 2, at least 3, at least 4, at least 5, at least 6, at least 7, at least 8, or at least 9.

23. The method of any one of claims 1-22, wherein the method reduces the fatigue score of the individual.

24. The method of claim 23, wherein the fatigue score is measured using the fatigue numeric rating scale (NRS).

25. The method of claim 24, wherein the method reduces the fatigue score by at least 1, at least 2, at least 3, at least 4, at least 5, at least 6, at least 7, at least 8, or at least 9.

26. The method of any one of claims 1-25, wherein administering the FcRn antagonist results in a reduction in the individual of serum IgG of at least 10%, at least 20%, at least 30%, at least 40%, at least 50%, at least 60%, at least 70%, at least 80%, or at least 90%.

27. The method of any one of claims 1-26, wherein administering the FcRn antagonist results in a reduction in serum IgG levels of from 60 to 80%.

28. The method of any one of claims 1-27, wherein administering the FcRn antagonist results in a reduction in serum IgG levels of about 70%.

29. The method of any one of claims 1-28, wherein serum from the individual produces a higher percentage of IgG-bound satellite glial cells (SGCs) or an increased SGC-binding intensity compared to serum from a healthy control patient.

30. The method of any one of claims 1-29, wherein administration of the FcRn antagonist results in a reduction of at least 10%, at least 20%, at least 30%, at least 40%, at least 50%, at least 60%, at least 70%, at least 80%, or at least 90% in the level of anti-SGC IgG in the individual.

31. The method of any one of claims 1-30, wherein the anti-FcRn antibody or FcRn-binding fragment thereof is administered at a dose of from 1 mg/kg to 50 mg/kg.

32. The method of any one of claims 1-31, wherein the anti-FcRn antibody or FcRn-binding fragment thereof is administered at a dose of from 4 mg/kg to 20 mg/kg.

33. The method of any one of claims 1-32, wherein the anti-FcRn antibody or FcRn-binding fragment thereof is administered at a dose of from 7 mg/kg to 10 mg/kg.

34. The method of any one of claims 1-33, wherein the anti-FcRn antibody or FcRn-binding fragment thereof is administered at a dose of 560 mg.

35. The method of any one of claims 1-34, wherein the anti-FcRn antibody or FcRn-binding fragment thereof is administered subcutaneously.

36. The method of any one of claims 1-35, wherein the anti-FcRn antibody or FcRn-binding fragment thereof is administered once a day, three times a week, twice a week, once a week, once every two weeks, once every three weeks, once every month, once every six weeks, once every two months, once every three months, once every four months, once every five months, or once every six months.

37. The method of any one of claims 1-36, wherein the anti-FcRn antibody or FcRn-binding fragment thereof is administered once a week.

38. The method of any one of claims 1-37, wherein the method further comprises administering a therapeutically effective amount of a second agent for treating FMS.

39. The method of claim 38, wherein the second agent is selected from the group consisting of an analgesic, an antidepressant, a dopamine agonist, an anti-seizure drug, an antihistamine, a hypnotic, a muscle relaxant, and an antipsychotic.

40. The method of claim 39, wherein the analgesic is selected from the group consisting of paracetamol (acetaminophen), ibuprofen, and opiates such as codeine, tramadol, morphine and oxycodone.

41. The method of claim 39, wherein the antidepressant is selected from the group consisting of a tricyclic antidepressant, a serotonin-noradrenaline reuptake inhibitor (SNRI) and a selective serotonin reuptake inhibitor (SSRI).

42. The method of claim 41, wherein the tricyclic antidepressant is selected from the group consisting of amitriptyline, desipramine, clomipramine, protriptyline, doxepin, imipramine, amoxapine, trazodone, nortriptyline, and trimipramine.

43. The method of claim 41, wherein the SNRI is selected from the group consisting of venlafaxine, duloxetine, desvenlafaxine, levomilnacipran, and milnacipran.

44. The method of claim 41, wherein the SSRI is selected from the group consisting of citalopram, escitalopram, fluoxetine, fluvoxamine, paroxetine, and sertraline.

45. The method of claim 39, wherein the anti-seizure drug is selected from the group consisting of gabapentin and pregabalin.

46. The method of claim 39, wherein the hypnotic is a benzodiazepine or nonbenzodiazopine.

47. The method of claim 46, wherein the benzodiazepine is selected from the group consisting of lorazepam, clonazepam, diazepam and alprazolam.

48. The method of claim 46, wherein the nonbenzodiazepine is selected from the group consisting of zolpidem, zaleplon, and eszopiclone.

49. The method of claim 39, wherein the muscle relaxant is selected from the group consisting of cyclobenzaprine, flexeril, orphenadrine citrate, tizanidine, and carisoprodol.

50. The method of claim 39, wherein the antipsychotic is quetiapine.

51. An FcRn antagonist for use in a method of treating or preventing fibromyalgia syndrome (FMS) in an individual, wherein the FcRn antagonist is an anti-FcRn antibody or FcRn-binding antibody fragment comprising:
(a) a heavy chain or heavy chain fragment having a variable region, wherein said variable region comprises three CDRs having the sequences given in SEQ ID NO: 4 for CDR H1, SEQ ID NO: 5 for CDR H2, and SEQ ID NO: 6 for CDR H3, and
(b) a light chain or light chain fragment having a variable region, wherein said variable region comprises three CDRs having the sequences given in SEQ ID NO: 9 for CDR L1, SEQ ID NO: 10 for CDR L2, and SEQ ID NO: 11 for CDR L3.

52. Use of an FcRn antagonist for the manufacture of a medicament for treating or preventing fibromyalgia syndrome (FMS) in an individual, wherein the FcRn antagonist is an anti-FcRn antibody or FcRn-binding antibody fragment comprising:
(a) a heavy chain or heavy chain fragment having a variable region, wherein said variable region comprises three CDRs having the sequences given in SEQ ID NO: 4 for CDR H1, SEQ ID NO: 5 for CDR H2, and SEQ ID NO: 6 for CDR H3, and
(b) a light chain or light chain fragment having a variable region, wherein said variable region comprises three CDRs having the sequences given in SEQ ID NO: 9 for CDR L1, SEQ ID NO: 10 for CDR L2, and SEQ ID NO: 11 for CDR L3.


SEQUENCE LISTING (24 sequences)

SEQ ID NO: 1 - protein, 116 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVS

SEQ ID NO: 2 - protein, 112 aa, synthetic construct [sequence omitted]

SEQ ID NO: 3 - protein, 112 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARYFDYWGQGTLVTVS

SEQ ID NO: 4 - protein, 10 aa, synthetic construct
GFTFSNYGMV

SEQ ID NO: 5 - protein, 17 aa, synthetic construct
YIDSDGDNTYYRDSVKG

SEQ ID NO: 6 - protein, 8 aa, synthetic construct
GIVRPFLY

SEQ ID NO: 7 - protein, 14 aa, Homo sapiens
YFDYWGQGTLVTVS

SEQ ID NO: 8 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 9 - protein, 16 aa, synthetic construct
KSSQSLVGASGKTYLY

SEQ ID NO: 10 - protein, 7 aa, synthetic construct
LVSTLDS

SEQ ID NO: 11 - protein, 9 aa, synthetic construct
LQGTHFPHT

SEQ ID NO: 12 - protein, 12 aa, Homo sapiens
YTFGQGTKLEIK

SEQ ID NO: 13 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 226 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 15 - protein, 447 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 357 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGTGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 19 - protein, 347 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 20 - protein, 346 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 21 - protein, 274 aa, Homo sapiens
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 22 - protein, 99 aa, Homo sapiens
IQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 23 - protein, 7 aa, synthetic construct
  VARIANT 6: note = 'X' may be any amino acid, preferably a hydrophobic amino acid
GHFGGXY

SEQ ID NO: 24 - protein, 4 aa, Homo sapiens
YFDY